(19) United States
(12) Patent Application Publication
(10) Pub. No.: US 2021/0018497 A1
(43) Pub. Date: Jan. 21, 2021

(54) BRET SENSOR MOLECULES FOR DETECTING HYDROLASES

(71) Applicant: Commonwealth Scientific and Industrial Research Organisation, Acton, ACT (AU)

(72) Inventors: Karine Caron, Acton (AU); Stephen Charles Trowell, Oxley (AU)

(21) Appl. No.: 16/639,331

(22) PCT Filed: Aug. 24, 2018
(86) PCT No.: PCT/AU2018/050906

§ 371 Date: Feb. 14, 2020

(30) Foreign Application Priority Data
Aug. 24, 2017 (AU) 2017903420

(51) Int. Cl. G01N 33/542; C12N 9/16; G01N 33/573; C12Q 1/44; C12Q 1/37; C12Q 1/42

(52) U.S. Cl. CPC G01N 33/542; C12N 9/16; G01N 33/573; G01N 2333/914; C12Q 1/37; C12Q 1/42; C12Q 1/44

(57) ABSTRACT

The present invention relates to bioluminescence resonance energy transfer sensor molecules having the structure R¹-L-R²—B or B—R²-L-R¹, wherein R¹ is a bioluminescent protein, L is a linking element, R² is a non-protein acceptor domain and B is a blocking group, and wherein R² bound to B comprises a hydrolysable bond which produces a change in BRET when hydrolysed. The invention also discloses a method of detecting a hydrolase by contacting a sample with a molecule B—R², then contacting with a compound R¹-L or L-R¹ under conditions to cause attaching of R² to L, and detecting a change in the BRET ratio. Specifically exemplified sensors comprise luciferase and fluorescein diacetate, which is hydrolysed by an esterase. The invention also discloses luciferase enzymes derived from RLuc8 by removing cysteine residues.

### FIELD OF THE INVENTION

The present invention relates to sensors and methods for detecting hydrolases, such as phosphatases, glycosidases, esterases, exopeptidases and lipases, in a sample. In particular, the present invention relates to sensors and methods for detecting hydrolases in food, beverages and in clinical samples. The sensors and methods may be used to determine the amount of hydrolase present in the sample.

### BACKGROUND OF THE INVENTION

Hydrolases are a class of enzymes found in all domains of life. Their roles vary, for example hydrolases are involved in the degradation of biomass, defence, pathogenesis and normal cell function. Assays for determining the activity of hydrolases, are routinely used in food, clinical and diagnostic settings. These assays often rely on spectrophotometric, amperometric, colorimetric or fluorescent detection. However, there is a growing need for assays which provide simple, sensitive and/or cost-effective alternatives to more traditional assay formats. There is also a need for reproducible assays that are suitable for high throughput screening. Of particular interest are sensors and assays that can be used to detect and measure the levels of a broad variety of hydrolytic enzymes, such as phosphatases, glycosidases, esterases, exopeptidases and lipases.

### SUMMARY OF THE INVENTION

The present inventors have identified sensor molecules that can be used to detect hydrolases in a sample. The present inventors have also identified methods of detecting a hydrolase in a sample.

In one aspect, there is provided a sensor molecule for detecting a hydrolase, the sensor molecule having a general formula selected from:

R¹-L-R²—B (I), or

B—R²-L-R¹ (II)

wherein

R¹ is a bioluminescent protein;

L is a linking element;

R² is a non-protein acceptor domain; and

B is a blocking group,

wherein R² bound to B comprises a hydrolysable bond and hydrolysis of the hydrolysable bond by the hydrolase produces a change in bioluminescence resonance energy transfer (BRET).

In an embodiment, the non-protein acceptor domain is a non-protein fluorescent acceptor domain.

In some embodiments, the blocking group stabilises the acceptor domain in a non-fluorescent state. In some embodiments, the blocking group stabilises the acceptor domain in a low fluorescent state.

In some embodiments, B comprises a phosphate containing moiety, sugar containing moiety, amino acid containing moiety, nucleotide, nucleoside, ester or ether.

In some embodiments, the linking element comprises an alkyl chain, glycol, ether, polyether, polyamide, polyester, peptide, polypeptide, amino acid or polynucleotide. In some embodiments, the linking element comprises a polypeptide. In some embodiments, R¹-L or L-R¹ are a single polypeptide. In some embodiments, the linking element comprises a cysteine residue and/or a lysine residue. In some embodiments, R² is attached to the linking element via the cysteine residue.

In some embodiments, R² is selected from an Alexa Fluor dye, Bodipy dye, Cy dye, fluorescein, dansyl, umbelliferone, fluorescent microsphere, luminescent microsphere, fluorescent nanocrystal, Marina Blue, Cascade Blue, Cascade Yellow, Pacific Blue, Oregon Green, Tetramethylrhodamine, Rhodamine, coumarin, BODIPY, resorufin, Texas Red, rare earth element chelates, or any combination or derivative thereof.

In some embodiments, the bioluminescent protein R¹ is selected from a luciferase, a β-galactosidase, a lactamase, a horseradish peroxidase, an alkaline phosphatase, a β-glucuronidase or a β-glucosidase. In some embodiments, R¹ is a luciferase comprising a Renilla luciferase, a Firefly luciferase, a Coelenterate luciferase, a North American glow worm luciferase, a click beetle luciferase, a railroad worm luciferase, a bacterial luciferase, a Gaussia luciferase, Aequorin, an Arachnocampa luciferase, or a biologically active variant or fragment of any one, or chimera of two or more, thereof.

In some embodiments, the bioluminescent protein, R¹, is capable of modifying a substrate. In some embodiments, the substrate is luciferin, calcium, coelenterazine, or a derivative or analogue of coelenterazine.

In some embodiments, the hydrolase is an esterase, lipase, protease, phosphatase, nuclease, glycosidase, DNA glycosylases and acid anhydride hydrolase. In some embodiments, the hydrolase is an esterase. In some embodiments, the hydrolase is a phosphatase. In some embodiments, the hydrolase is a lipase.

In some embodiments, R¹ comprises RLuc8, L is a polypeptide comprising a cysteine residue, and R² bound to B is fluorescein diacetate. In this embodiment, R² bound to B is attached to the cysteine via a maleamide linking group, L comprises 28 amino acids and L-R¹ is a single polypeptide. This sensor may be used as an esterase sensor.

In some embodiments, the separation and relative orientation of R¹ and R², in the presence and/or the absence of hydrolase, is within ±50% of the Förster distance. In some embodiments, the Förster distance of R¹ and R² is at least 4.0 nm. In some embodiments, the Förster distance of R¹ and R² is at least 5.6 nm. In some embodiments, the Förster distance of R¹ and R² is between about 4.0 nm and about 10 nm. In some embodiments, the Förster distance of R¹ and R² is between about 5.6 nm and about 10 nm.

In another aspect, there is provided a method of detecting a hydrolase in a sample, the method comprising

i) contacting a sample with the sensor molecule defined herein; and

ii) detecting a change in BRET ratio, wherein the change in the BRET ratio corresponds to the presence of a hydrolase in the sample.

In yet another aspect, there is provided, a method of detecting a hydrolase in a sample, the method comprising:

i) contacting a sample with a blocked non-protein acceptor domain having the structure B—R² to form a treated sample;

ii) contacting the treated sample with a compound of formula R¹-L or L-R¹ under conditions to cause attaching of R² to L; and

iii) detecting a change in BRET ratio, wherein the change in the BRET ratio corresponds to the presence of a hydrolase in the sample and the formation of a compound of formula R¹-L-R² or R²-L-R¹, and wherein

R¹ is a bioluminescent protein;

L is a linking element;

R² is a non-protein acceptor domain; and

B is a blocking group and R² bound to B comprises a hydrolysable bond.

In an embodiment, the non-protein acceptor domain R² is a non-protein fluorescent acceptor domain.

In some embodiments, R² comprises a cysteine specific electrophile or an amine specific electrophile. In some embodiments, L comprises a cysteine and/or a lysine residue.

In some embodiments, the methods defined herein further comprise determining the concentration and/or activity of the hydrolase in the sample. In some embodiments, the methods defined herein are performed on a microfluidic device.

In some embodiments, the sample is selected from the group consisting of air, liquid, biological material and soil. In some embodiments, the sample may be any suitable biological material, such as (but not limited to) milk, blood, serum, sputum, mucus, pus, urine, sweat, faeces, tears or peritoneal fluid. In some embodiments, the sample comprises a biological material selected from the group consisting of milk, blood, serum, sputum, mucus, pus and peritoneal fluid. In some embodiments, the sample may be a suspension or extract obtained by washing, soaking, grinding or macerating a solid agricultural, food or other substance in an aqueous solution and using the liquid phase. The liquid phase may be clarified by settling, filtration or centrifugation.

In yet another aspect there is provided a variant bioluminescent protein comprising at least one less cysteine residue when compared to the corresponding naturally occurring protein. In some embodiments, the variant bioluminescent protein lacks a cysteine residue at a position corresponding to amino acid number 24, 73 and/or 124 of RLuc (SEQ ID NO: 49). In some embodiments, the variant bioluminescent protein lacks a cysteine residue at a position corresponding to amino acid number of 24 RLuc. In some embodiments, the variant bioluminescent protein lacks a cysteine residue at a position corresponding to amino acid number of 73 RLuc. In some embodiments, the variant bioluminescent protein lacks a cysteine residue at a position corresponding to amino acid number of 124 RLuc. In some embodiments, the variant bioluminescent protein lacks a cysteine residue at a position corresponding to amino acid numbers 24 and/or 73 of RLuc8. In some embodiments, the variant bioluminescent protein lacks a cysteine residue at a position corresponding to position 24 or position 73 of RLuc8 (SEQ ID NO: 50). In some embodiments, the variant bioluminescent protein lacks a cysteine residue at a position corresponding to position 24 and position 73 of RLuc8 (SEQ ID NO: 50). In some embodiments, the variant bioluminescent protein lacks a cysteine residue at a position corresponding to amino acid number 24, 73 and/or 124 of RLuc2 (SEQ ID NO: 51).

In yet another aspect there is provided a polynucleotide encoding the variant bioluminescent protein defined herein.

In some embodiments, there is provided a vector comprising the polynucleotide encoding the variant bioluminescent protein defined herein.

In some embodiments, there is provided a host cell comprising the polynucleotide and/or the vector defined herein.

In some embodiments, there is provided a process for producing a variant bioluminescent protein, the process comprising cultivating a host cell defined herein or a vector defined herein under conditions which allow expression of the polynucleotide encoding the protein, and recovering the expressed protein.

In some embodiments, there is provided a sensor molecule as defined herein, wherein R¹ is the variant bioluminescent protein as defined herein.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1—Illustrative sensor molecule for detecting a hydrolase as defined herein. In the illustrated embodiment, the bioluminescent protein, R¹, is linked by a linking element, L, to a non-protein acceptor domain, R², whose fluorescence is modulated by an acetate blocking group, B. In the illustrated embodiment, the blocking group, B, is removed by an esterase which restores the fluorescence of the non-protein acceptor domain and allows BRET to occur.

FIG. 2—Examples of cysteine specific labelling strategies using Michael acceptors such as maleimide, acrylamide and phenylcarbonylacrylamide.

FIG. 3—Optimisation of labelling conditions to minimise internal RLuc8 labelling. 5 μM R¹-L (RLuc8 with a linking element) was incubated with 4 eq. of fluorescein-5-maleimide (20 μM) in 50 mM MES buffer, pH 5.0, at 4° C. and the reaction monitored using BRET before the addition of fluorescein-5-maleimide and at 6, 15, 30 and 60 minutes after the addition of fluorescein-5-maleimide. (A) wt-RLuc8 (SEQ ID NO: 1); (B) RLuc8Cys1 (SEQ ID NO: 2).

FIG. 4—(A) Comparison of Bioluminescence Resonance Energy Transfer (BRET) for an illustrative sensor molecule (RLuc8Cys2-fluorescein-diacetate) (solid line) and the sensor molecule after incubation with esterase (0.8 U) for 30 min at 37° C. (B) Comparison of BRET for an illustrative sensor molecule (solid line) and an unblocked sensor molecule (RLuc8Cys2-fluorescein).

FIG. 5—Comparison of BRET ratio for the RLuc8Cys1-FM conjugate at pH 7.0, 7.5 and 8.0 (1 μM conjugate in 50 mM HEPES, 50 mM NaCl) (mean±S. D., n=3).

FIG. 6—(A) Introduction of a single cysteine residue on the N-terminal peptide linking element of RLuc8 at one of either position 1 (1 amino acid between R¹ and R², SEQ ID NO: 2), 2 (11 amino acids between R¹ and R², SEQ ID NO: 3) or 3 (21 amino acids between R¹ and R², SEQ ID NO: 4); (B) BRET ratios of 1 μM of the sensor molecule (mean±S. D., n=6).

FIG. 7—(A) Comparison of BRET for RLuc8Cys1, RLuc8Cys2, RLuc8Cys3, RLuc8Cys4, RLuc8Cys5 and MBP(K239C)RLuc8 labelled with fluorescein-maleimide (mean±S. D., n=3). (B) Comparison of BRET for RLuc8Cys1, RLuc8Cys2, RLuc8Cys3, RLuc8Cys4, RLuc8Cys5 and MBP(K239C)RLuc8 labelled with fluorescein-maleimide (FM) or Rhodamine Red C2 maleimide (RM) (mean±S. D., n=3). (C) Comparison of the BRET² ratio for RLuc8Cys1, RLuc8Cys2, RLuc8Cys3, RLuc8Cys4, RLuc8Cys5 and MBP(K239C)RLuc8 labelled with fluorescein-maleimide (mean±S. D., n=3).

FIG. 8—Use of illustrative sensor molecules, RLuc8Cys4-fluorescein-diacetate, RLuc8Cys3-fluorescein-diacetate and RLuc8Cys2-fluorescein-diacetate, to detect and measure esterase activity at pH 7.0 and 25° C.

FIG. 9—Use of an illustrative sensor molecule (RLuc8Cys4-fluorescein-diacetate) to detect and measure esterase activity at 30° C. (A), 25° C. (B) or 20° C. (C).

FIG. 10—Method for detecting hydrolase according to embodiments of the present disclosure. (A) In this embodiment, the small-molecule acceptor is covalently attached to the BRET donor prior to contact with the hydrolase. (B) In this embodiment, the small-molecule acceptor, R², is pre-activated with the hydrolase before being covalently attached to the BRET donor for detection.

### KEY TO THE SEQUENCE LISTING

SEQ ID NO: 1—wt-RLuc8 (comprises RLuc8 and N-terminal linking element).

SEQ ID NO: 2—RLuc8Cys1 (comprises RLuc8 and N-terminal linking element).

SEQ ID NO: 3—RLuc8Cys2 (comprises RLuc8 and N-terminal linking element).

SEQ ID NO: 4—RLuc8Cys3 (comprises RLuc8 and N-terminal linking element).

SEQ ID NO: 5 to 7—Linking element sequences.

SEQ ID NO: 8—wt-RLuc8(C24X).

SEQ ID NO: 9—wt-RLuc8(C73Z).

SEQ ID NO: 10—wt-RLuc8(C24X.C73Z).

SEQ ID NO: 11—Nucleotide sequence encoding wt-RLuc8.

SEQ ID NO: 12—Nucleotide sequence encoding RLuc8Cys1.

SEQ ID NO: 13—Nucleotide sequence encoding RLuc8Cys2.

SEQ ID NO: 14—Nucleotide sequence encoding RLuc8Cys3.

SEQ ID NO: 15 to 20—Primer sequences.

SEQ ID NO: 21 to 22—High affinity substrate for mTG.

SEQ ID NO: 23—Sortase recognition sequence.

SEQ ID NO: 24 to 30—Spacer sequences.

SEQ ID NO: 31—High affinity substrate for mTG.

SEQ ID NO: 32—RLuc8Cys4 (comprises RLuc8 and N-terminal linking element).

SEQ ID NO: 33—RLuc8Cys5 (comprises RLuc8 and N-terminal linking element).

SEQ ID NO: 34—MBP(K239C)RLuc8 (comprises N-terminal linking element comprising MBP(K239C) and RLuc8).

SEQ ID NO: 35—Nucleotide sequence encoding RLuc8Cys4.

SEQ ID NO: 36—Nucleotide sequence encoding RLuc8Cys5.

SEQ ID NO: 37—Nucleotide sequence encoding MBP(K239C)RLuc8.

SEQ ID NO: 38 to 43—Primer sequences.

SEQ ID NO: 44 to 48—Linking element containing a cysteine residue.

SEQ ID NO: 49—Amino acid sequence of RLuc.

SEQ ID NO: 50—Amino acid sequence of RLuc8.

SEQ ID NO: 51—Amino acid sequence of RLuc2.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, BRET based sensor technology, bioconjugation techniques, protein chemistry, biochemistry and the like).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, even more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Sensor

Throughout the specification “sensor” and “sensor molecule” are used interchangeably.

In one aspect, the present disclosure provides a sensor molecule for detecting a hydrolase, the sensor molecule having a general formula selected from:

R¹-L-R²—B (I), or

B—R²-L-R¹ (II)

wherein

R¹ is a bioluminescent protein;

L is a linking element;

R² is a non-protein acceptor domain; and

B is a blocking group,

wherein R² bound to B comprises a hydrolysable bond and hydrolysis of the hydrolysable bond by the hydrolase produces a change in bioluminescence resonance energy transfer (BRET).

In some embodiments, R¹-L or L-R¹ are a single polypeptide. In some embodiments, R¹-L is a continuous stretch of amino acids. In other embodiments, L-R¹ is a continuous stretch of amino acids. For example, the bioluminescent protein (R¹) and the linking element are a single stretch of amino acids such as, but not limited to, a bioluminescent protein covalently attached to the N-terminus of the linking element or a bioluminescent protein covalently attached to the C-terminus of the linking element. The covalent attachment is a peptide bond. For example, R¹-L or L-R¹ are a single polypeptide which comprises a polypeptide sequence selected from SEQ ID NO: 1, SEQ ID NO: 2, SEQ ID NO: 3, SEQ ID NO: 4, SEQ ID NO: 32 and SEQ ID NO: 33. In some embodiments, the single polypeptide can also comprise a polypeptide sequence having at least 30%, 35%, 40%, 45%, 50%, 55%, 60%, 65%, 70%, 75%, 80%, 85%, 90%, 95%, 96%, 97%, 98%, 99% or 100% sequence identity to any one or more of SEQ ID NO: 1, SEQ ID NO: 2, SEQ ID NO: 3, SEQ ID NO: 4, SEQ ID NO: 32 and SEQ ID NO: 33.

In a further embodiment, there is also provided a nucleic acid which comprises a polynucleotide sequence encoding R¹-L or L-R¹ as defined herein. In some embodiments, the nucleic acid is an isolated nucleic acid. For example, in one embodiment the nucleic acid molecule comprises a sequence encoding the polypeptide sequence selected from the group consisting of SEQ ID NO: 1, SEQ ID NO: 2, SEQ ID NO: 3, SEQ ID NO: 4, SEQ ID NO: 32 and SEQ ID NO: 33. In some embodiments, the nucleic acid molecule comprises a sequence encoding a polypeptide sequence having at least 30%, 35%, 40%, 45%, 50%, 55%, 60%, 65%, 70%, 75%, 80%, 85%, 90%, 95%, 96%, 97%, 98%, 99% or 100% sequence identity to any one or more of SEQ ID NO: 1, SEQ ID NO: 2, SEQ ID NO: 3, SEQ ID NO: 4, SEQ ID NO: 32 and SEQ ID NO: 33. In one embodiment the nucleic acid molecule comprises a sequence encoding the polypeptide sequence selected from the group consisting of SEQ ID NO: 1, SEQ ID NO: 2, SEQ ID NO: 3 and SEQ ID NO: 4 or a polypeptide sequence having at least 30%, 35%, 40%, 45%, 50%, 55%, 60%, 65%, 70%, 75%, 80%, 85%, 90%, 95%, 96%, 97%, 98%, 99% or 100% sequence identity to any one or more of SEQ ID NO: 1, SEQ ID NO: 2, SEQ ID NO: 3 and SEQ ID NO: 4. In addition to the sequence encoding the sensor of the invention (or part thereof), the nucleic acid molecule may contain other sequences such as primer sites, transcription factor binding sites, vector insertion sites and sequences which resist nucleolytic degradation (e.g. polyadenosine tails). The nucleic acid molecule may be DNA or RNA and may include synthetic nucleotides, provided that the polynucleotide is still capable of being translated in order to synthesize a protein of the invention.

In some embodiments, the nucleic acid forms part of a vector such as a plasmid. In addition to the nucleic acid sequence described above, the plasmid comprises other elements such as a prokaryotic origin of replication (for example, the E. coli OR1 origin of replication) an autonomous replication sequence, a centromere sequence; a promoter sequence capable of expressing the nucleic acid in the host cell which is operably linked to the nucleic acid, a terminator sequence located downstream of the nucleic acid sequence, an antibiotic resistance gene and/or a secretion signal sequence. A vector comprising an autonomous replication sequence is also a yeast artificial chromosome. In some alternative embodiments, the vector is a virus, such as a bacteriophage and comprises, in addition to the nucleic acid sequence of the invention, nucleic acid sequences for replication of the bacteriophage, such as structural proteins, promoters, transcription activators and the like.

The nucleic acid or vector of the invention may be used to transfect or transform host cells in order to synthesize the sensor or target sequence of the invention. Suitable host cells include prokaryotic cells such as E. coli and eukaryotic cells such as yeast cells, or mammalian or plant cell lines. Host cells are transfected or transformed using techniques known in the art such as electroporation; calcium phosphate base methods; a biolistic technique or by use of a viral vector.

After transfection/transformation, the nucleic acid or vector of the invention is transcribed as necessary and translated. In some embodiments, the synthesized protein is extracted from the host cell, either by virtue of its being secreted from the cell due to, for example, the presence of secretion signal in the vector, or by lysis of the host cell and purification of the protein therefrom.

In some embodiments, the sensor (or part thereof, for example R¹-L or L-R¹) is provided as a cell-free composition. As used herein, the term “cell free composition” refers to an isolated composition which contains few, if any, intact cells and which comprises the sensor. Examples of cell free compositions include cell (such as yeast cell) extracts and compositions containing an isolated, purified and/or recombinant sensor molecules (such as proteins). Methods for preparing cell-free compositions from cells are well-known in the art.

Blocking Group

In the sensors of the present invention “B” refers to a blocking group and B bound to R² comprises a hydrolysable bond. B is capable of modulating the fluorescence properties of R² such that the fluorescence properties of R² bound to B when the hydrolysable bond is intact are different to the fluorescence properties of R² bound to B when the hydrolysable bond has been cleaved. In some embodiments, the blocking group B stabilises the acceptor domain R² in fluorescent state A. Cleavage of the hydrolysable bond of R²—B or B—R² by a hydrolase changes the fluorescent state of the acceptor domain R² to fluorescent state A*. Fluorescent state A and fluorescent state A* are different such that cleavage of the hydrolysable bond results in a change in BRET. In some embodiments, B is selected such that R² bound to B has a reduced signal relative to the signal of R² without B.

In some embodiments, blocking group B changes the absorption spectrum of R² such that the intensity of the light emitted by R² upon addition of a substrate differs between fluorescent state A and fluorescent state A* and cleavage of the hydrolysable bond results in a change in BRET. For example, cleavage of the hydrolysable bond by a hydrolase may increase the intensity of light emitted by R². This can occur when the acceptor domain is a quencher and the blocking group changes the fluorescent properties of the acceptor domain so that it no longer acts as a quencher. Cleavage of the hydrolysable bond results in the acceptor domain being returned to a quencher and a decrease in BRET. Alternatively, cleavage of the hydrolysable bond by a hydrolase may decrease the intensity of light emitted by R². In some embodiments, the blocking group B stabilises the acceptor domain R² in a low-fluorescent state. In some embodiments, the blocking group B stabilises the acceptor domain R² in a non-fluorescent state. After cleavage of the hydrolysable bond by a hydrolase, R² is no longer in the low or non-fluorescent state. Consequently, cleavage of the hydrolysable bond by the hydrolase results in a change in BRET that may be detected and/or quantified.

As used herein, a “low-fluorescent state” refers to a fluorescent state that is distinguishable from that of the high-fluorescent state. For example, a low-fluorescent state may be at least 20% less fluorescent, at least 30% less fluorescent, at least 40% less fluorescent, at least 50% less fluorescent, at least 60% less fluorescent, at least 70% less fluorescent, at least 80% less fluorescent, at least 90% less fluorescent, at least 95% less fluorescent, at least 98% less fluorescent or at least 99% less fluorescent than R² when not bound to B. In some embodiments, the low-fluorescent state is at least 90% less fluorescent, at least 95% less fluorescent, at least 98% less fluorescent or at least 99% less fluorescent than R² when not bound to B. In some embodiments, the low-fluorescent state is between 20-99%, 30-99%, 40-99%, 50-99%, 60-99%, 70-99%, 80-99% or 90-99%, less fluorescent than R² when not bound to B. In some embodiments, the low-fluorescent state is between 80-99% less fluorescent, 85-97% less fluorescent or between 90-95% less fluorescent than R² when not bound to B.

As used herein, a “non-fluorescent state” refers to a fluorescent state that is 100 times the level of the background noise, 50 times the level of the background noise, 20 times the level of the background noise, 10 times the level of the background noise or 5 times the level of the background noise. For example, a fluorophore in a “non-fluorescent” state may exhibit near baseline excitation and emission. As the person skilled in the art would appreciate “non-fluorescent state” and “low-fluorescent state” are not mutually exclusive.

A blocked fluorophore in a low-fluorescent or non-fluorescent state may also be referred to as a masked or latent fluorophore.

In some embodiments, blocking group B changes the absorption spectrum of R² such that the peak wavelength of the absorption spectrum differs between fluorescent state A and fluorescent state A* and cleavage of the hydrolysable bond results in a change in BRET. For example, in some embodiments, the blocking group B changes the absorption spectrum of R² such that R² emits little or no light upon excitation. In these embodiments, there can be energy transfer between R¹ and R² but R² functions as a quencher until the hydrolysable bond is cleaved. In other words, in the presence of a substrate for R¹ the uncleaved sensor is dark due to the action of R²—B. Once the hydrolysable bond is cleaved by a hydrolase, R² no longer functions as a quencher and emits light on excitation. Consequently, upon addition of the substrate for R¹, an increase in fluorescence emission from R² (and corresponding change in BRET) may be detected and/or quantified. In other embodiments, the blocking group B changes the absorption spectrum of R² such that there is no, or substantially no, overlap with the emission spectrum of R¹ and there is no, or substantially no, energy transfer between R¹ and R². After cleavage of the hydrolysable bond by a hydrolase, the fluorescent state of R² changes such that absorption spectrum of R² overlaps (at least partially) with the emission spectrum of R¹ and there is energy transfer between R¹ and R². Consequently, upon addition of the substrate for R¹, an increase in fluorescence emission from R² (and corresponding change in BRET) may be detected and/or quantified.

In some embodiments, blocking group B changes the emission spectrum of R² such that the emission spectrum differs between fluorescent state A and fluorescent state A* and cleavage of the hydrolysable bond results in a change in BRET. For example, cleavage of the hydrolysable bond by a hydrolase may increase the intensity of light emitted by R². Alternatively, cleavage of the hydrolysable bond by a hydrolase may decrease the intensity of light emitted by R². In some embodiments, the blocking group B stabilises the acceptor domain R² in a low-fluorescent state or non-fluorescent state. After cleavage of the hydrolysable bond by a hydrolase, R² is no longer in the low-fluorescent state or non-fluorescent state. Consequently, cleavage of the hydrolysable bond by the hydrolase results in a change in BRET may be detected and/or quantified.

In alternative embodiments, blocking group B according to the present disclosure may act as a quencher and decreases the intensity of light emitted by the BRET pair, R¹ and R², by accepting energy emitted as a result of the activity of the BRET pair without re-emitting it as light energy. In these embodiments, there can be energy transfer between R¹ and R², and R² and B but B functions as a quencher until the hydrolysable bond is cleaved. In other words, in the presence of a substrate for R¹ the sensor is dark due to the action of B. Once the hydrolysable bond is cleaved by a hydrolase, B is removed and the BRET pair emits light on excitation. Consequently, upon addition of the substrate for R¹ a change in BRET may be detected and/or quantified.

B can be any suitable blocking group known to a person in the art and can be selected by the person skilled on the art based on the hydrolase of interest. A suitable blocking group is a group that is capable of modulating the fluorescent properties of R². B or B bound to R² comprises a substrate for a hydrolase. For example, in some embodiments, B comprises a hydrolysable bond. In some embodiments, B is attached to R² via a hydrolysable bond.

In the context of the present disclosure, B comprises a phosphate containing moiety, sugar containing moiety, amino acid containing moiety, amide containing moiety, nucleotide, nucleoside, ester or ether. In some embodiments, B comprises a phosphate containing moiety, sugar containing moiety, or ester. In some embodiments, B comprises a phosphate containing moiety or ester. In some embodiments, B comprises an ester. As the person skilled in the art would appreciate a blocking group B can be classified as one or more of the above. For example, a nucleotide is both a phosphate containing moiety and a nucleotide.

In some embodiments, B can comprise a phosphate containing moiety. In some embodiments, B comprises a phosphate ester moiety comprising one or more covalently bound phosphate groups. For example, B comprises a phosphate ester moiety having the following structure:

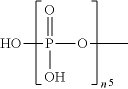

where n is an integer between 1-4. In one set of embodiments, B is H₂PO₄—. As an example, B comprises or is attached to R² by a phosphoester bond. In these embodiments, the sensor can form a substrate for a phosphatase.

In alternative embodiments, B comprises an amino acid containing moiety. For example, in some embodiments B comprises (Xₐₐ)ₙ where Xₐₐ is an amino acid and n is an integer from 1 to 10, 2 to 9, 3 to 7, 4 to 6 or 5. In some embodiments, B comprises a cleavage site for a protease, for example B contains at least the preferred P1-P1′ amino acids for the protease (Schechter and Berger, 1967; Schechter and Berger, 1968). In these embodiments, the sensor can form a substrate for a protease.

In other embodiments, B comprises an amide bond or is attached to R² via an amide bond. For example, B may be selected from the group consisting of the following structures:

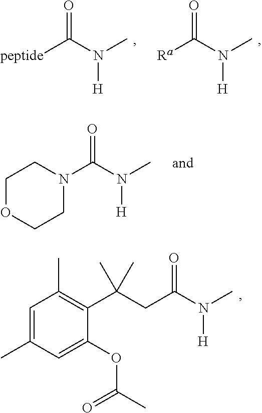

wherein R^{a} comprises a (Xₐₐ)ₙ where Xₐₐ is an amino acid and n is an integer from 1 to 10, 2 to 9, 3 to 7, 4 to 6 or 5. In these embodiments, the sensor can form a substrate for a protease or a hydrolase which acts on non-peptidic C—N bonds.

In other embodiments, B comprises a sugar containing moiety. In an example, B comprises or is attached to R² by a glycosidic bond. As used herein, a glycosidic bond is a covalent bond that that joins a carbohydrate (sugar) molecule to another group, which may or may not be another carbohydrate. In some embodiments, the glycosidic bond is an O-glycosidic bond, an S-glycosidic bond or an N-glycosidic bond. For example, B comprises a glucose moiety, a galactose moiety or a fructose moiety. In these embodiments, the sensor can form a substrate for a glycosidase, such as an α-glycosidase or a β-glycosidase.

In some embodiments, B comprises a nucleoside. A nucleoside comprises a nitrogenous base and a 5-carbon sugar. In some embodiments, the 5-carbon sugar is ribose. In some embodiments, the 5-carbon sugar is deoxyribose. In some embodiments, the nitrogenous base is selected from the group consisting of adenine (A), uracil (U), guanine (G), thymine (T), and cytosine (C). In some embodiments, the nucleoside is selected from the group consisting of cytidine, uridine, adenosine, guanosine, thymidine and inosine. In some embodiments, the nucleoside is selected from the group consisting of deoxycytidine, deoxyuridine, deoxyadenosine, deoxyguanosine, deoxythymidine and deoxyinosine. In these embodiments, the sensor can form a substrate for a nucleoside hydrolase.

In some embodiments, B comprises a nucleotide. As used herein, a nucleotide is defined broadly and comprises at least one phosphate group, a nitrogenous base and a 5-carbon sugar. In some embodiments, the 5-carbon sugar is ribose. In some embodiments, the 5-carbon sugar is deoxyribose. In some embodiments, the nitrogenous base is selected from the group consisting of adenine (A), uracil (U), guanine (G), thymine (T), and cytosine (C). For example, in some embodiments, a nucleotide is a nucleoside and at least one phosphate group, for example, but not limited to, a nucleoside monophosphate, a nucleoside diphosphate, and a nucleoside triphosphate. In some embodiments, B comprises a linear nucleotide such as ATP, GTP, CTP and UTP. In some embodiments, B comprises a cyclic nucleotide such as cyclic guanosine monophosphate (cGMP) and cyclic adenosine monophosphate (cAMP). In some embodiments, B is selected from the group consisting of coenzyme A, flavin adenine dinucleotide (FAD), flavin mononucleotide (FMN), nicotinamide adenine dinucleotide (NAD) and nicotinamide adenine dinucleotide phosphate (NADP⁺). In these embodiments, the sensor can form a substrate for a N-glycosyl hydrolase or nucleotide hydrolase.

In some embodiments, B comprises an oligonucleotide. For example, B can comprise (X_{NT})ₙ where X_{NT} is a nucleotide and n is an integer from 1 to 10, 2 to 9, 3 to 7, 4 to 6 or 5. In some X_{NT} comprises a nitrogenous base selected from the group consisting of adenine (A), uracil (U), guanine (G), thymine (T), and cytosine (C). In these embodiments, the sensor can form a substrate for a nuclease or a DNA glycosylase.

In some embodiments, B comprises an ester. In some embodiments, B is selected from the group consisting of the following structures:

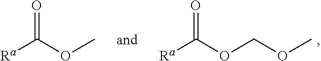

where R^{a} is C₁₋₃₀ straight or branched chain alkyl, optionally substituted with one of more halogen atoms, C₃₋₈ cycloalkyl or cycloalkenyl, C₃₋₈heterocyclyl or aryl. In some embodiments, R^{a} is C₁₋₄ straight or branched chain alkyl, optionally substituted with one of more halogen atoms. In some embodiments, R^{a} is a methyl, ethyl, butyl, propyl, butyl or t-butyl. In some embodiments, R^{a} is a methyl. In these embodiments, the sensor can form a substrate for an esterase. In some preferred embodiments, B comprises one or more groups having the following structure where R^{a} is as defined herein:

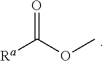

Preferably R^{a} is a methyl.

In some embodiments, B comprises a thioester. In some embodiments, B is selected from the group consisting of the following structures:

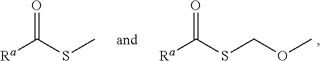

where R^{a} is C₁₋₃₀ straight or branched chain alkyl, optionally substituted with one of more halogen atoms, C₃₋₈ cycloalkyl or cycloalkenyl, C₃₋₈heterocyclyl or aryl. In some embodiments, R^{a} is C₁₋₄ straight or branched chain alkyl, optionally substituted with one of more halogen atoms. In some embodiments, R^{a} is a methyl, ethyl, butyl, propyl, butyl or t-butyl. In these embodiments, the sensor can form a substrate for a thioesterase.

In some embodiments, B comprises an ether or a thioether. In some embodiments, B is selected from the group consisting of the following structures:

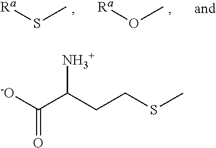

where R^{a} is C₁₋₃₀ straight or branched chain alkyl, optionally substituted with one of more halogen atoms, C₃₋₈ cycloalkyl or cycloalkenyl, C₃₋₈heterocyclyl or aryl. In some embodiments, R^{a} is C₁₋₄ straight or branched chain alkyl, optionally substituted with one of more halogen atoms. In some embodiments, R^{a} is a methyl, ethyl, butyl, propyl, butyl or t-butyl. In these embodiments, the sensor can form a substrate for a dealkylase.

In some embodiments, B comprises a halogen or a haloalkyl. In some embodiments, B is

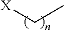

where n is an integer from 1-8 and X is a halogen. In some embodiments, X is selected from the group consisting of Cl, Br, F and I. In these embodiments, the sensor can form a substrate for a dehalogenase.

In some embodiments, B comprises a β-lactam. In some embodiments, comprises a β-lactam antibiotic such as a penicillin, cephalosporin, cephamycin, or carbapenem. In some embodiments, B comprises a β-lactam antibiotic selected from the group consisting of penicillins and cephalosporins. For example, in some embodiments B comprises the following structure:

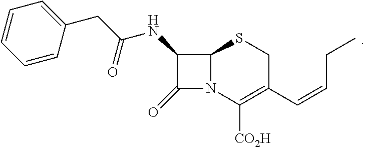 [SMILES: CC/C=C\C1=C(C(=O)O)N2C(=O)[C@@H](NC(=O)Cc3ccccc3)[C@H]2SC1]

In these embodiments, the sensor can form a substrate for a β-lactamase.

In some embodiments, B comprises a “trimethyl lock” As used herein a “trimethyl lock” is an o-hydroxy-cinnamic acid derivative. In these embodiments, B bound to R² is often referred to as a “latent fluorophore”, “masked fluorophore” or “pro-fluorophore” and is in a low-fluorescent state or non-fluorescent state. An example of a sensor with a trimethyl lock has the following structure:

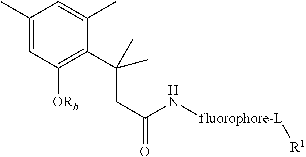

wherein the fluorophore is any suitable fluorophore that can be linked to the trimethyl lock, and wherein OR_{b} comprises a hydrolysable bond that can be hydrolysed by the hydrolase of interest to unmask the phenolic oxygen. For example, R_{b} may be an acyl group, a phosphoryl group, a sulphuryl group or a glycosyl group. In one example, R_{b} is an acetyl group. Depending on the fluorophore, the sensor may comprise more than one “trimethyl lock”. Once the phenolic oxygen is unmasked, unfavourable steric interactions between the three methyl groups lead to rapid lactonization, release of the fluorophore bound to L-R¹ and an increase in the BRET ratio. Suitable fluorophores include, but are not limited to, rhodamine 110, 7-amino-4-methylcoumarin and cresyl violet. In this example, the sensor can form a substrate for an esterase. In another example, OR_{b} is an OPO₃H₂ group and the sensor can form a substrate for a phosphatase. Example latent fluorophores based on the trimethyl lock are provided in Chandran et al., 2005, Levine and Raines, 2012; Lavis et al., 2006a and Lavis et al., 2006b. Without wishing to be bound by theory, it is thought that including the trimethyl block between the hydrolysable bond and the fluorophore may improve the accessibility of the hydrolysable bond to the hydrolase.

In some embodiments, B comprises a self-immolative linker. The self-immolative linker may be located between the fluorophore and hydrolysable bond potentially improving the accessibility of the hydrolysable bond to the hydrolase. As used herein, a “self-immolative linker” is a reversible covalent connection between two molecular species (in this case a fluorophore and a hydrolysable bond). Prior to cleavage of the hydrolysable bond, the fluorophore is in a low-fluorescent or non-fluorescent state. Self-decomposition of the covalent connector is triggered by cleavage of the hydrolysable bond by the hydrolase releasing the fluorophore in a high-fluorescent state. Accordingly, cleavage of the hydrolysable bond by a hydrolase increases the BRET ratio. Suitable self-immolative fluorogenic probes are described in Ż [symbol] dło-Dobrowolska et al., 2016.

Hydrolysable Bond

The sensors of the present invention comprise a hydrolysable bond. As used herein, a “hydrolysable bond” is a covalent bond that can be broken by a hydrolase. In other words, a hydrolysable bond is a substrate for a hydrolase. Cleavage of the hydrolysable bond changes the fluorescent properties of R² resulting in a change in BRET. B or B bound to R² comprises a hydrolysable bond. In some embodiments, B comprises the hydrolysable bond. In other embodiments, B is bound to R² by the hydrolysable bond.

The invention provides the use of any suitable hydrolysable bond in the sensors of the present disclosure. In some examples, the hydrolysable bond is selected from the group consisting of an ester bond, amide (or peptide) bond, an ether bond, a thioether bond, a glycosidic bond, an thioester bond, a phosphate ester bond, a carbon-nitrogen bond, an acid anhydride bond, a carbon-carbon bond, a halide bond, a phosphorous-nitrogen bond, a sulphur-nitrogen bond, a carbon-phosphorous bond, a sulphur-sulphur bond and a carbon-sulphur bond. In some embodiments, the hydrolysable bond is selected from the group consisting of an ester bond, amide (or peptide) bond, an ether bond, a thioether bond, a glycosidic bond, a thioester bond, a phosphate ester bond and a carbon-nitrogen bond. In preferred embodiments, the hydrolysable bond is an ester bond.

In embodiments of the present disclosure, R²—B/B—R₂ comprises a hydrolysable bond and forms a substrate for the hydrolase of interest. Suitable non-limiting examples of R²—B/B—R² are listed in Table 1.

TABLE 1
Non-limiting examples of R² bound to B.

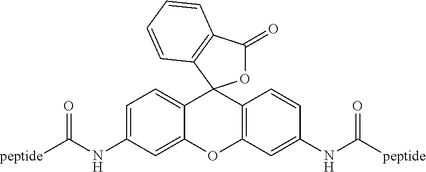

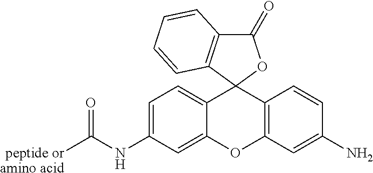

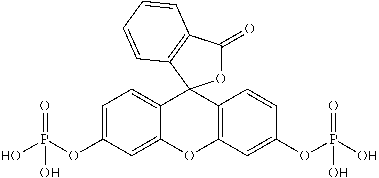 [SMILES: O=C1OC2(c3ccc(OP(=O)(O)O)cc3Oc3cc(OP(=O)(O)O)ccc32)c2ccccc21]

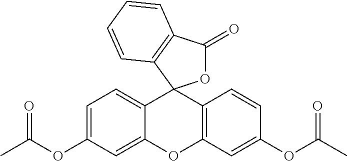 [SMILES: CC(=O)Oc1ccc2c(c1)Oc1cc(OC(C)=O)ccc1C21OC(=O)c2ccccc21]

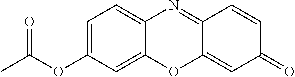 [SMILES: CC(=O)Oc1ccc2nc3ccc(=O)cc-3oc2c1]

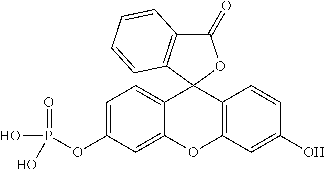 [SMILES: O=C1OC2(c3ccc(O)cc3Oc3cc(OP(=O)(O)O)ccc32)c2ccccc21]

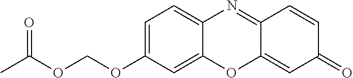 [SMILES: CC(=O)OCOc1ccc2nc3ccc(=O)cc-3oc2c1]

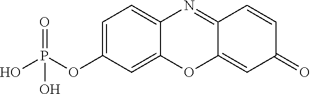 [SMILES: O=c1ccc2nc3ccc(OP(=O)(O)O)cc3oc-2c1]

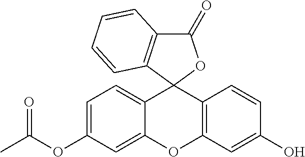 [SMILES: CC(=O)Oc1ccc2c(c1)Oc1cc(O)ccc1C21OC(=O)c2ccccc21]

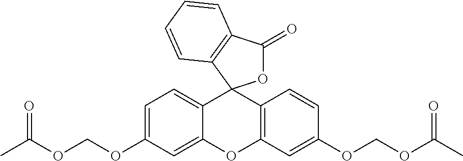 [SMILES: CC(=O)OCOc1ccc2c(c1)Oc1cc(OCOC(C)=O)ccc1C21OC(=O)c2ccccc21]

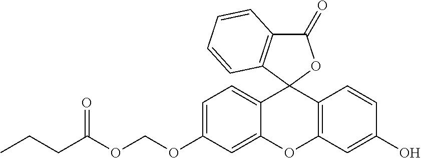 [SMILES: CCCC(=O)OCOc1ccc2c(c1)Oc1cc(O)ccc1C21OC(=O)c2ccccc21]

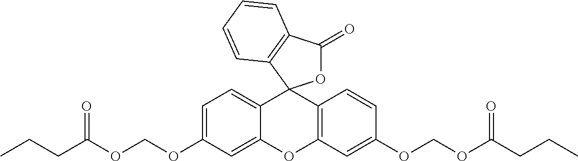 [SMILES: CCCC(=O)OCOc1ccc2c(c1)Oc1cc(OCOC(=O)CCC)ccc1C21OC(=O)c2ccccc21]

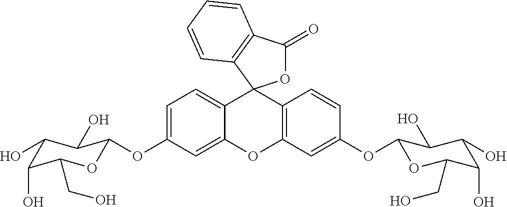 [SMILES: O=C1OC2(c3ccc(OC4OC(CO)C(O)C(O)C4O)cc3Oc3cc(OC4OC(CO)C(O)C(O)C4O)ccc32)c2ccccc21]

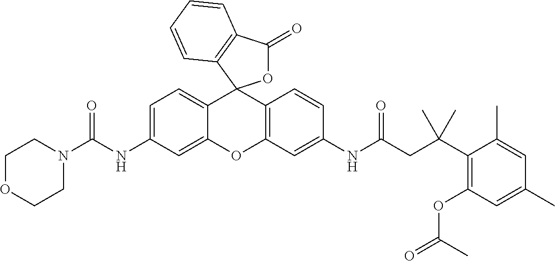 [SMILES: CC(=O)Oc1cc(C)cc(C)c1C(C)(C)CC(=O)Nc1ccc2c(c1)Oc1cc(NC(=O)N3CCOCC3)ccc1C21OC(=O)c2ccccc21]

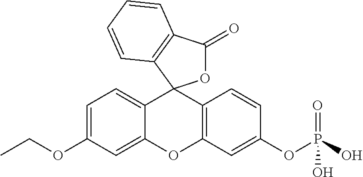

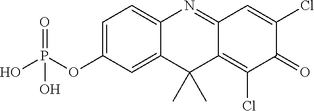 [SMILES: CC1(C)C2=C(Cl)C(=O)C(Cl)=CC2=Nc2ccc(OP(=O)(O)O)cc21]

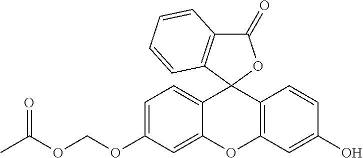 [SMILES: CC(=O)OCOc1ccc2c(c1)Oc1cc(O)ccc1C21OC(=O)c2ccccc21]

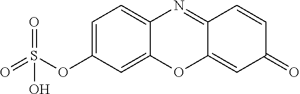 [SMILES: O=c1ccc2nc3ccc(OS(=O)(=O)O)cc3oc-2c1]

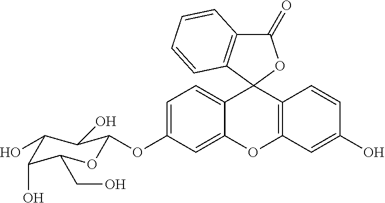 [SMILES: O=C1OC2(c3ccc(O)cc3Oc3cc(OC4OC(CO)C(O)C(O)C4O)ccc32)c2ccccc21]

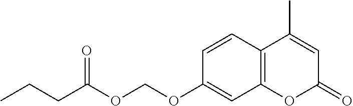 [SMILES: CCCC(=O)OCOc1ccc2c(C)cc(=O)oc2c1]

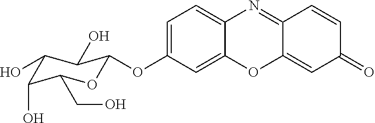 [SMILES: O=c1ccc2nc3ccc(OC4OC(CO)C(O)C(O)C4O)cc3oc-2c1]

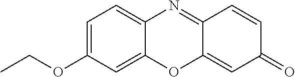 [SMILES: CCOc1ccc2nc3ccc(=O)cc-3oc2c1]

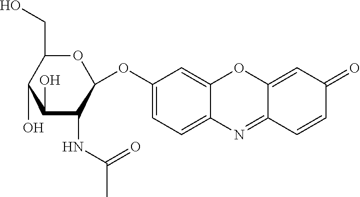

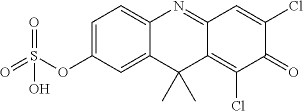 [SMILES: CC1(C)C2=C(Cl)C(=O)C(Cl)=CC2=Nc2ccc(OS(=O)(=O)O)cc21]

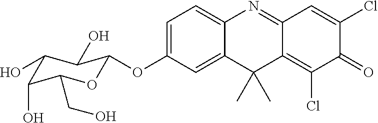 [SMILES: CC1(C)C2=C(Cl)C(=O)C(Cl)=CC2=Nc2ccc(OC3OC(CO)C(O)C(O)C3O)cc21]

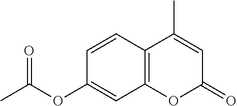 [SMILES: CC(=O)Oc1ccc2c(C)cc(=O)oc2c1]

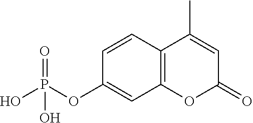 [SMILES: Cc1cc(=O)oc2cc(OP(=O)(O)O)ccc12]

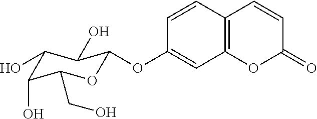 [SMILES: O=c1ccc2ccc(OC3OC(CO)C(O)C(O)C3O)cc2o1]

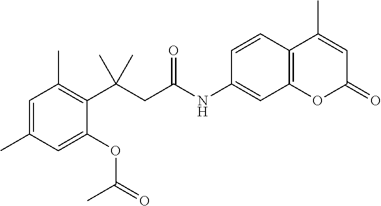 [SMILES: CC(=O)Oc1cc(C)cc(C)c1C(C)(C)CC(=O)Nc1ccc2c(C)cc(=O)oc2c1]

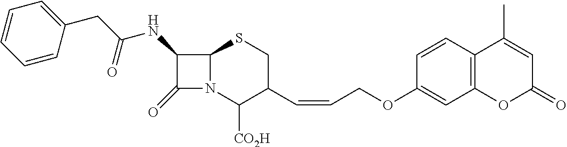

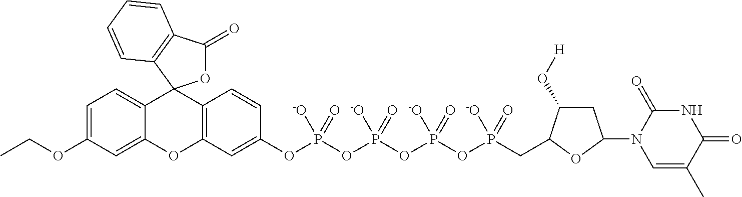

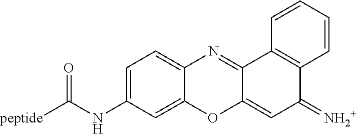

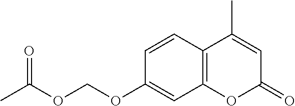 [SMILES: CC(=O)OCOc1ccc2c(C)cc(=O)oc2c1]

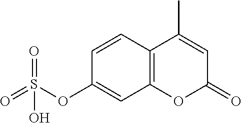 [SMILES: Cc1cc(=O)oc2cc(OS(=O)(=O)O)ccc12]

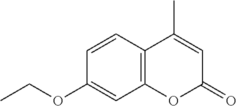

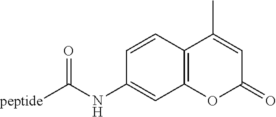

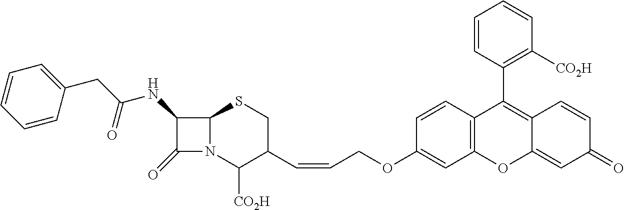

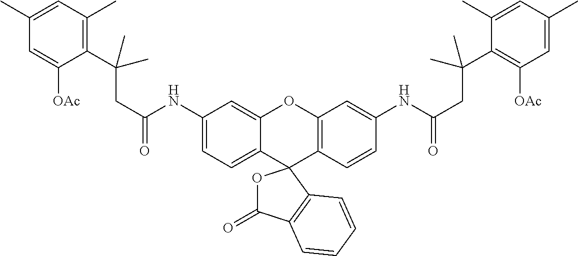

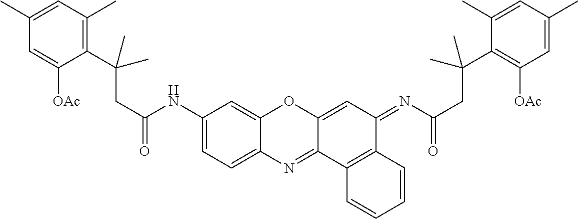

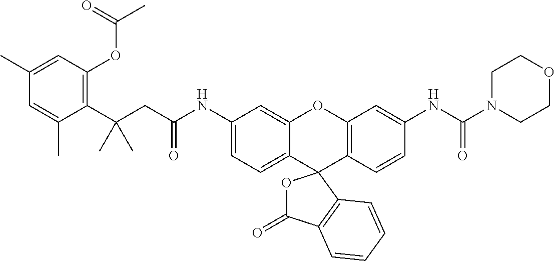 [SMILES: CC(=O)Oc1cc(C)cc(C)c1C(C)(C)CC(=O)Nc1ccc2c(c1)Oc1cc(NC(=O)N3CCOCC3)ccc1C21OC(=O)c2ccccc21]

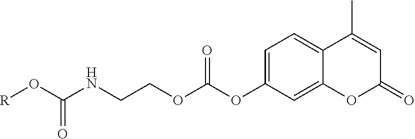

R = CH₂C₆H₅
R = CH(CH₃)₃
R = CH₂CH═CH₂

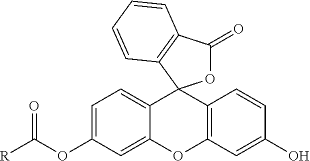

R = (CH₂)₁₀CH₃

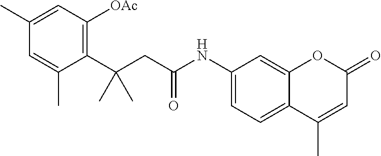 [SMILES: CC(=O)Oc1cc(C)cc(C)c1C(C)(C)CC(=O)Nc1ccc2c(C)cc(=O)oc2c1]

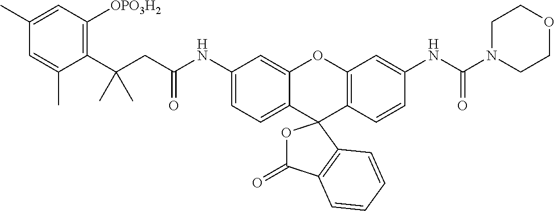

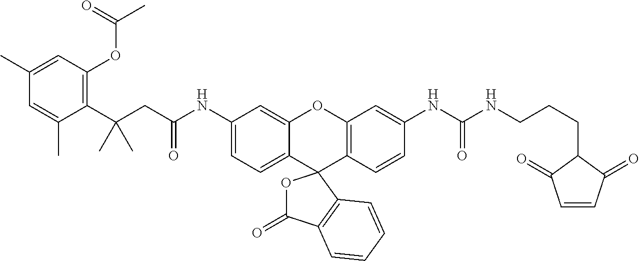 [SMILES: CC(=O)Oc1cc(C)cc(C)c1C(C)(C)CC(=O)Nc1ccc2c(c1)Oc1cc(NC(=O)NCCCC3C(=O)C=CC3=O)ccc1C21OC(=O)c2ccccc21]

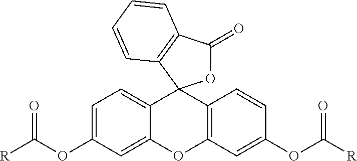

R = (CH₂)₁₀CH₃

In preferred embodiments, R²—B/B—R₂ comprises fluorescein acetate or fluorescein diacetate.

The above compounds are available from commercial suppliers, may be synthesised according to be methods known in the art or may be synthesised according to published methods.

Linking Element

The sensors of the present invention comprise a linking element, L. The linking element is a molecular moiety that attaches R¹ to R². In some embodiments, the linking element (or part thereof) is an integral part of R¹ (for example, the N- or C-terminus of R¹ or a naturally occurring cysteine or lysine residue in R¹ such that R² is directly bound to R¹ via the side-chain of the naturally occurring cysteine or lysine). In some embodiments, the linking element (or part thereof) is an integral part of R² (for example, an amine or thiol group in R² such that R¹ is directly bound to R² via the amine or thiol group or a sortase recognition sequence). In some embodiments the linking element is a separate chemical entity which attaches R¹ to R².

Suitable linking elements include, but are not limited to, polypeptides, polynucleotides, polyalkylene glycol, polyalkylene glycol where at least one oxygen of the polyalkylene glycol chain is substituted with nitrogen, polyamine (Herve et at, 2008), peptide nucleic acid (PNA) (Egholm et al., 2005), locked nucleic acid (LNA) (Singh et al., 1998), triazoles, piperazines, oximes, thiazolidines, aromatic ring systems, alkanes, alkenes, alkynes, cyclic alkanes, cyclic alkenes, amides, thioamides, ethers, and hydrazones. In some embodiments, the linking element comprises or is selected from the group consisting of alkyl chain, glycol, polyglycol, ether, polyether, polyamide, polyester, amino acid, peptide, polypeptide or polynucleotide. In some embodiments, the linking element is a peptide or polypeptide. In some embodiments, the linking element is polyethylene glycol or polypropylene glycol.

The length of the linking element depends on the linking element selected, as well as the R¹-R² pair selected. For example, the length of the linking element depends on the working distance range of the R¹-R² pair selected. In some embodiments, the length of the linking element can be varied to alter or control the change in BRET ratio.

In some embodiments, the linking element can comprise polyalkylene glycol. Suitable polyalkylene glycols include polyethylene glycol (PEG) and methoxypolyethylene glycol (mPEG). PEG is a polymer of ethylene glycol and, depending on substitutions, can have the chemical formula C₂ₙ₊₂H₄ₙ₊₆Oₙ₊₂. For example, the linking element comprises PEG having up to about 40 ethylene glycol moieties. In some embodiments, the linking element comprises a PEG linker having up to about 30 ethylene glycol moieties. In some embodiments, the linking element comprises a PEG linker having up to about 20 ethylene glycol moieties. In some embodiments, the linking element comprises a PEG linker having up to about 10 ethylene glycol moieties. In some embodiments, the linking element comprises a PEG linker having up to about 8 ethylene glycol moieties. In some embodiments, the linking element comprises a PEG linker having up to about 6 ethylene glycol moieties. Other useful polyalkylene glycols are polypropylene glycols, polybutylene glycols, PEG-glycidyl ethers, and PEG-oxycarbonylimidazole.

In alternative embodiments, the linking element comprises an oligonucleotide. The oligonucleotide can comprise both nucleoside bases or modified nucleoside bases or both. The linking element can have up to about 50 nucleoside bases and/or modified nucleoside bases. In one embodiment, the linking element can have up to about 40 nucleoside bases and/or modified nucleoside bases. In another embodiment the linking element comprises up to about 30 nucleoside bases and/or modified nucleoside bases. In another embodiment the linking element comprises up to about 20 nucleoside bases and/or modified nucleoside bases. In another embodiment the linking element comprises up to about 10 nucleoside bases and/or modified nucleoside bases. In yet another embodiment, the linking element comprises up to about 5 nucleoside bases and/or modified nucleoside bases.

In preferred embodiments, the linking element comprises a polypeptide. Peptide, oligopeptide and polypeptide are used interchangeably herein to refer to a polymer of two or more amino acids. Typically, oligopeptide is used for chains containing between 2 and 10 amino acids and the term polypeptide is used for chains containing more than 10 amino acids. The peptide can comprise naturally or unnaturally occurring amino acids or a combination thereof. The peptide or polypeptide can comprise modified amino acids. In one embodiment, the linking element can have up to about 50 amino acid residues. In one embodiment, the linking element can have up to about 40 amino acid residues. In another embodiment the linking element comprises up to about 37 amino acid residues. In another embodiment the linking element comprises up to about 31 amino acid residues. In another embodiment the linking element comprises up to about 30 amino acid residues. In another embodiment the linking element comprises up to about 28 amino acid residues. In another embodiment the linking element comprises up to about 23 amino acid residues. In another embodiment the linking element comprises up to about 21 amino acid residues. In another embodiment the linking element comprises up to about 20 amino acid residues. In another embodiment the linking element comprises up to about 13 amino acid residues. In another embodiment the linking element comprises up to about 11 amino acid residues. In another embodiment the linking element comprises up to about 10 amino acid residues. In yet another embodiment, the linking element comprises up to about 5 amino acid residues. In yet another embodiment, the linking element comprises up to about 3 amino acid residues. In yet another embodiment, the linking element comprises 1 amino acid. In yet another embodiment, the linking element comprises between about 1-30 amino acids, about 5-25 amino acids, about 7-23 amino acids, about 10-20 amino acids, or about 13-18 amino acids. In yet another embodiment, the linking element comprises between about 1-30 amino acids, about 10-30 amino acids, about 20-30 amino acids, about 25-30 amino acids, or about 28 amino acids. In preferred embodiments, the linking element comprises about 25-30 amino acids, or about 28 amino acids. In preferred embodiments, the linking element comprises a free cysteine or a free lysine. As used herein, “free” when defining to an amino acid refers to an unmodified side-chain, for example one with a-SH group or —NH₂/—NH₃⁺ group respectively. In some embodiments, the linking element is a peptide sequence at the N-terminus of R¹. In some embodiments, the linking element is a peptide sequence at the C-terminus of R¹.

In some embodiments, the linking element is a peptide comprising the sequence C. In some embodiments, the linking element is a peptide comprising the sequence CDDKDRWGSEF (SEQ ID NO: 5). In some embodiments, the linking element is a peptide comprising the sequence CQQMGRDLYDDDDKDRWGSEF (SEQ ID NO: 6). In some embodiments, the linking element is a peptide comprising the sequence MRGSHHHHHHGMASMTGGQQMGRDLYDDDDKDRWGSEF (SEQ ID NO: 7). In some embodiments, at least one amino acid in the sequence is replaced by a cysteine. For example, at least one of the amino acids in SEQ ID NO: 5, SEQ ID NO: 6 or SEQ ID NO: 7 is replaced by a cysteine. In some embodiments, the linking element comprises or consists of the sequence provided in any one of SEQ ID NO: 44, SEQ ID NO: 45, SEQ ID NO: 46, SEQ ID NO: 47 and SEQ ID NO: 48.

In some embodiments, the linking element comprises a high affinity Gln substrate for microbial transglutaminase (Oteng-Pabi et al., 2014). For example, the linking element comprises a peptide having the sequence selected from the group consisting of WALQRPH (SEQ ID NO: 21) and WELQRPY (SEQ ID NO: 22). In some embodiments, the linking element comprises a sortase recognition sequence (Theile et al., 2013). For example, the linking element comprises a peptide having the sequence LPXT, where X is any amino acid (SEQ ID NO: 23). As the person skilled in the art would understand, sortase mediated reactions can be used to label the N-terminus of R¹. In some embodiments, the linking element further comprises a spacer sequence. In some embodiments, the spacer sequence comprises one or more glycine, serine and/or threonine residues. For example, in some embodiments, the spacer sequence comprises an amino acid sequence selected from GSSGGS (SEQ ID NO: 24), GGSGGS (SEQ ID NO: 25), GGTGGG (SEQ ID NO: 26), GGGGGT (SEQ ID NO: 27), LQGGTGGG (SEQ ID NO: 28), FEGGTGGG (SEQ ID NO: 29) and GGSGGSL (SEQ ID NO: 30).

The linking element may have a mass of less than about 5 kD, less than about 4.5 kD, less than about 4.0 kD, less than about 3.5 kD, less than about 3 kD, or less than about 2.5 kD, and can be less than about 2 kD. The linking element may have a mass of between about 1 kDa and 5 kD, between about 2 kDa and about 5 kD, and between about 3 KDa and about 5 kD.

In some embodiments, the linking element comprises a reactive moiety. The reactive moiety can react with a chemical group in R¹ and/or R² by any means of chemical reaction to form the sensor molecules described herein. Any suitable reactive moiety may be used. In some embodiments, the reactive moiety is selected from the group consisting of a sulfhydryl reactive moiety, an amine reactive moiety and a carbonyl reactive moiety. In some embodiments, the reactive moiety is a group which reacts with a sulfhydryl reactive moiety, an amine reactive moiety and/or a carbonyl reactive moiety. For example, the reactive moiety may include of a free cysteine residue, free lysine residue or a carbonyl group.

For example, in some embodiments, the linking element is provided with a sulfhydryl reactive moiety which is reactive with a free cysteine (e.g., a naturally occurring cysteine or a cysteine introduced by mutation) in R¹ and/or R² to form a covalent linkage therebetween. In other embodiments, the linking element is provided with an amine reactive moiety which is reactive with a lysine residue (e.g., a naturally occurring lysine or a lysine introduced by mutation) in R¹ and/or R² to form a covalent linkage therebetween. In other embodiments, the linking element is provided with a carbonyl reactive moiety which is reactive with a carbonyl group in R¹ and/or R² to form a covalent linkage therebetween. In still another embodiment, the linking element is provided with a free cysteine or a free lysine which is reactive with a sulfhydryl reactive moiety in R² and/or R¹ to form a covalent linkage therebetween. In yet another embodiment, the linking element is provided with a free lysine which is reactive with an amine reactive moiety in R² and/or R¹ to form a covalent linkage therebetween. In another embodiment, the linking element is provided with a carbonyl group which is reactive with a carbonyl reactive moiety in R² and/or R¹ to form a covalent linkage therebetween.

Sulfhydryl reactive moieties include thiol, triflate, tresylate, aziridine, oxirane, S-pyridyl, maleimidobenzoyl sulfosuccinimide ester, or maleimide moieties. Preferred sulfhydryl reactive moieties include maleimide, acrylamide, phenylcarbonylacrylamide and iodoacetamide Amine reactive moieties include active esters (including, but not limited to, succinimidyl esters, sulfosuccinimidyl esters, tetrafluorophenyl esters, and sulfodichlorophenol esters), isothiocyanates, dichlorotriazines, aryl halides, acyl azides and sulfonyl chlorides. Of these amine reactive moieties, active esters are preferred reagents as they produce stable carboxamide bonds (see, for example, Banks and Paquette, 1995). Carbonyl reactive moieties include primary amines such as hydrazides and alkoxyamines Carbonyl containing moieties include aldehydes (RCHO) and ketones (RCOR′). In some examples, the aldehyde is created by periodate-oxidation of a sugar group in the linking element.

In one example, when the linking element comprises PEG (or NPEG) it may also comprise one or more reactive moieties, such as an electrophilic or nucleophilic group (for example, see WO 2007/140282), which can be used to attach the PEG linker to R¹ and/or R². In some embodiments, the linking element is derived from a PEG-diacid or an NPEG-diacid. In these embodiments, the carboxyl group of the PEG-diacid or an NPEG-diacid linking element is linked to the terminal amino group of a terminal residue of R¹ via an amide bond. The other carboxyl group of the PEG-diacid or an NPEG-diacid linking element is linked via an amide bond to R².

In one example, when the linking element comprises a peptide it may also comprise cysteine residue and/or a lysine residue. In preferred embodiments, the linking element comprises a cysteine.

A person skilled in the art would appreciate that the length of the linker can impact BRET between the bioluminescent protein and the acceptor domain. Accordingly, the preferred length of the linker can vary depending on the bioluminescent protein and the acceptor domain used in the sensor.

Non-Protein Acceptor Domain (R²)

R² can be any suitable non-protein acceptor domain. As used herein, an “acceptor domain” is any molecule that is capable of accepting energy emitted as a result of the activity of the bioluminescent protein, R¹ (as described herein). In some embodiments, the non-protein acceptor domain can be a fluorescent acceptor domain or a quencher. As used herein, the term “fluorescent acceptor domain” (also referred herein to as “fluorescent acceptor molecule”) refers to any compound which can accept energy emitted as a result of the activity of the bioluminescent protein, R¹, and re-emit it as light energy. As used herein, the term “quencher” refers to any compound which can accept energy emitted as a result of the activity of the bioluminescent protein, R¹, without re-emitting it as light energy. A non-fluorescent acceptor can be a quencher.

There are many acceptor domains that can be employed in this invention. Suitable acceptor domains are non-proteinaceous and include organic molecules, such that in preferred embodiments R² is an organic acceptor domain. In preferred embodiments, the acceptor domain is not a quantum dot.

In some embodiments, R² is a non-protein fluorescent acceptor domain Any suitable non-protein fluorescent acceptor domain can be used. In some embodiments, R² is selected from the group consisting of Alexa Fluor dye, Bodipy dye, Cy dye, fluorescein, dansyl, umbelliferone, Marina Blue, Cascade Blue, Cascade Yellow, Pacific Blue, Oregon Green, Tetramethylrhodamine, Rhodamine, coumarin, boron-dipyrromethene (BODIPY), resorufin, Texas Red, rare earth element chelates, or any combination or derivatives thereof. Examples of derivatives include, but are not limited to, amine reactive derivatives, aldehyde/ketone reactive derivatives, cytosine reactive or sulfhydryl reactive derivatives.

In some embodiments, R² is fluorescein or a derivative thereof. Suitable derivatives include, but are not limited to, amine-reactive fluorescein derivatives, fluorescein isothiocyanate (FITC), NHS-fluorescein, NHS-LC-fluorescein, sulfhydryl-reactive fluorescein derivatives, 5-(and 6)-iodoacetamido-fluorescein, fluorescein-5-maleimide, fluorescein-6-maleimide, SAMSA-fluorescein, aldehyde/ketone and cytosine reactive fluorescein derivatives, fluorescein-5-thiosemicarbazide and 5-(((2-(carbohydrazine)methyl)thio) acetyl)-aminofluorescein. In some embodiments, R² is a fluorescein-5-maleimide derivative. In some embodiments, R² is a fluorescein-6-maleimide derivative. In preferred embodiments, B—R² or R²—B is fluorescein-diacetate-6-maleimide. In preferred embodiments, B—R² or R²—B is fluorescein-diacetate-5-maleimide.

In some embodiments, R² is rhodamine or a derivative thereof. Suitable derivatives include, but are not limited to, amine-reactive rhodamine derivatives, tetramethylrhodamine-5-(and 6)-isothiocyanate, NHS-rhodamine, Lissamine™ rhodamine B sulfonyl chloride, Lissamine™ rhodamine B sulfonyl hydrazine, sulphydryl-reactive rhodamine derivatives, tetramethylrhodamine-5-(and 6)-iodoacetamide, aldehyde/ketone and cytosine reactive rhodamine derivatives, Texas red hydrazine and texas red sulfonyl chloride. In some embodiments, R² is a sulforhodamine B, C₂ maleimide derivative (also referred to as (RhodamineRed™ C2-maleimide or 2-(6-(diethylamino)-3-(diethyliminio)-3H-xanthen-9-yl)-5-(N-(2-(2,5-dioxo-2,5-dihydro-1H-pyrrol-1-yl)ethyl)sulfamoyl)benzenesulfonate).

In some embodiments, R² is coumarin or a derivative thereof. Suitable derivatives include, but are not limited to, amine-reactive coumarin derivatives, AMCA, AMCA-NHS, AMCA-sulfo-NHS, sulphydryl-reactive coumarin derivatives, AMCA-HPDP, DCIA, aldehyde and ketone reactive coumarin derivatives and AMCA-hydrazide.

In some embodiments, R² is boron-dipyrromethene (BODIPY) or a derivative thereof. Suitable derivatives include, but are not limited to, amine-reactive boron-dipyrromethene dyes, BODIPY FL C₃-SE, BODIPY 530/550 C₃, BODIPY 530/550 C₃-SE, BODIPY 530/550 C₃-hydrazide, BODIPY 493/503 C₃-hydrazide, BODIPY FL C₃-hydrazide, sulphydryl-reactive boron-dipyrromethene dyes, BODIPY FL 1A, BOPDIY 530/550 1A, Br-BOPDIPY 493/503 and aldehyde and ketone reactive boron-dipyrromethene dyes.

In some embodiments, R² is Cy (cyanine) dye or a derivative thereof. Suitable derivatives include, but are not limited to, amine reactive cyanine dyes, thiol-reactive cyanine dyes and carbonyl-reactive cyanine dyes.

In some embodiments, R² is a quencher. Any suitable quencher can be used. In some embodiments, R² is selected from the group consisting of DABCYL [4-((4-(Dimethylamino) phenyl)azo)benzoic acid], DABSYL (Dimethylaminoazosulfonic acid), metal nanoparticles such as gold and silver, black hole quenchers (BHQ), QSY dyes and QXL quenchers. In some embodiments, R² is selected from the group consisting of DABCYL [4-((4-(Dimethylamino) phenyl)azo)benzoic acid], DAB SYL (Dimethylaminoazosulfonic acid), black hole quenchers (BHQ), QSY dyes and QXL quenchers.

In some embodiments, R² can be attached to the linking element L to form the sensor via a reactive moiety naturally occurring in R¹, a reactive moiety naturally occurring in the linking element L, by adding a coupling group to the linking element L and/or by a coupling group present in R². For example, in some embodiments the linking element comprises a cysteine such that R² can be attached to the linking element L via the thiol containing side-chain of the cysteine. In some embodiments the linking element comprises a lysine such that R² can be attached to the linking element L via the amine containing side-chain of the lysine. In some embodiments, the linking element L comprises a non-natural amino acid such that R² can be attached to the linking element L via the side-chain of the non-natural amino acid. In some embodiments, the linking element L comprises a sugar group such that R² can be attached to the linking element L via hydrazide reaction chemistry or alkoxyamine reaction chemistry.

Exemplary coupling groups are described hereinafter, and methods for incorporating such coupling groups into the linking element L for attaching to R² or R¹ or into R² or R¹ for attaching to the linking element L are known to the person skilled in the art. For example, suitable coupling groups and associated techniques are described and explained in Greg T. Hermanson, Bioconjugate Techniques (Third Edition), Academic Press (2013). In the case of compounds of the invention comprising a protected functional moiety or a protected coupling group, removal of the protective group is performed by methods known in the art. As used in this context, the term “attaching” refers to the formation of a covalent bond between the linking group L and R¹ and/or L and R².

Suitable coupling groups include, but are not limited to, cysteine specific electrophiles and/or amine specific electrophiles. In some embodiments, and one or more of R¹, R² and the linking element comprises a cysteine specific electrophile. Any cysteine specific electrophile known to the person skilled in the art can be used. For example, cysteine specific electrophiles include, but are not limited to, maleimides, alkyl halides, aryl halides, α-halocarbonyls (e.g. iodoacetamides), pyridyl disulfides, acrylamides and phenyl carbonyl acrylamides. Other thiol specific coupling groups include, but are not limited to, haloacetyl and alkylhalide derivatives, aziridines, acryloyl derivatives, arylating agents, thiol-disulphide exchange reagents, vinyl sulfone derivatives, metal thiol dative bonds, native chemical ligation, cisplatin modification of methionine and cysteine.

In some embodiments, the cysteine specific electrophiles are Michael acceptors such as maleimide, acrylamide and phenylcarbonylacrylamide which are shown in FIG. 2. In some embodiments, R¹ can be directly bound to the Michael acceptor or indirectly bound to the Michael acceptor via linkage chemistry. Examples of suitable linkage chemistries include, but are not limited to, C₁₋₁₀ alkylene straight or branched chain comprising from 0-4 backbone (i.e., non-substituent) heteroatoms, optionally substituted with from 1 to 4 substituents independently selected from the group consisting of C₁₋₆ alkyl straight or branched chain, —NO₂, —NH₂, ═O, halogen, trihalomethyl, C₁₋₆ alkoxy, —OH, —CH₂OH, and —C(O)NH₂. In preferred embodiments, the cysteine specific electrophiles are maleimides which are linked according to the reaction scheme:

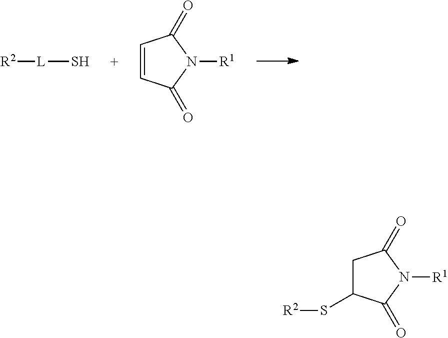

where R²-L-SH comprises a free thiol, either as a free thiol or following deprotection of a protected thiol.

Any amine specific electrophile known to the person skilled in the art can be used. For example, amine specific electrophiles include, but are not limited to, activated esters, sulfonyl chlorides and isothiocyanates. Other amine specific coupling groups include, but are not limited to, isocyanates, acyl azides, N-hydroxysuccinimide (NHS) esters, tosylate esters, aldehydes and glycoxals, epoxides and oxiranes, carbonates, arylating agents, imidoesters, carbodiimides, ahydrides, fluorophenyl esters, hydroxymethylphosphine derivatives and guanidination of amines.

Preferred amine specific electrophiles include imidoester and NHS esters. NHS esters yield stable products upon reaction with primary or secondary amines Coupling is efficient at physiological pH, and NHS-ester cross-linkers are more stable in solution than their imidate counterparts. Primary amines are the principle targets for NHS-esters. Accessible α-amine groups present on the N-termini of proteins can react with NHS-esters to form amides. The s-amino group of lysine reacts significantly with NHS-esters. A covalent amide bond is formed when the NHS-ester cross-linking agent reacts with primary amines, releasing N-hydroxysuccinimide.

Other suitable techniques can be used to attach R² to L. For example, carbodiimides can be used to couple carboxyls to primary amines or hydrazides, resulting in formation of amide or hydrazone bonds. Carbodiimides are unlike other coupling agents in that no cross-bridge is formed between the carbodiimide and the molecules being coupled; rather, a peptide bond is formed between an available carboxyl group and an available amine group. Depending on availability, carboxy termini of proteins can be targeted, as well as glutamic and aspartic acid side chains. In another example, reductive alkylation using aldehydes in the presence of sodium cyanoborohydride can be used to attach R² to L.

In some embodiments, R² can be attached to L via enzyme mediated labelling. Suitable enzymes include, but are not limited to, sortases and transglutaminases. Sortases can affect site-specific N-terminal labelling of proteins (Theile et al., 2013). Transglutaminases affect site-specific labelling of glutamine specific residues (Oteng-Pabi et al., 2014). For example, for sortase mediated N-terminal labelling R² comprises a coupling group comprising a peptide having the sequence LPXTZ, where X is any amino acid and Z is glycine or alanine (SEQ ID NO: 31).

Other techniques include, but are not limited to, native chemical ligation, Diels-Alder reagent pairs, hydrazine-aldehyde reagent pairs, aminooxy-aldehyde reagent pairs, click chemistry and Staudinger ligation. These techniques are described in more detail in Greg T. Hermanson, Bioconjugate Techniques (Third Edition), Academic Press (2013).

While coupling groups have been defined here based on functional group specificity, the person skilled in the art would be aware that these coupling groups have the potential to react with functional groups other than the one intended. For example, while N-hydroxysuccinimide esters are defined herein as an amine specific coupling group, they can also react with cysteine, histidine, serine, threonine, and tyrosine side-chain groups. Similarly, while maleimides are defined herein as being a cysteine specific electrophile they can also react with amines under the right conditions.

Bioluminescent Protein (R¹)

Bioluminescence is a form of chemiluminescence. Chemiluminescence is the emission of energy with limited emission of heat (luminescence), as the result of a chemical reaction. Chemiluminescence emission occurs as the energy from the excited states of organic dyes, which are chemically induced, decays to ground state. The duration and the intensity of the chemiluminescence emission are mostly dependent on the extent of the chemical reagents present in the reaction solution. Non-enzymatic chemiluminescence is the result of chemical reactions between an organic dye and an oxidizing agent in the presence of a catalyst. Bioluminescence relies upon the activity of an enzyme, often referred to as a bioluminescent protein. As used herein, the term “bioluminescent protein” refers to any protein capable of acting on a suitable substrate to generate luminescence.

It is understood in the art that a bioluminescent protein is an enzyme which converts a substrate into an activated product which then releases energy as it relaxes. The activated product (generated by the activity of the bioluminescent protein on the substrate) is the source of the bioluminescent protein-generated luminescence that is transferred to the acceptor molecule.

Exemplary bioluminescent proteins are described hereinafter (see, for example, Table 2). Light-emitting systems have been known and isolated from many luminescent organisms including bacteria, protozoa, coelenterates, molluscs, fish, millipedes, flies, fungi, worms, crustaceans, and beetles, particularly click beetles of genus Pyrophorus and the fireflies of the genera Photinus, Photuris, and Luciola. Additional organisms displaying bioluminescence are listed in WO 00/024878, WO 99/049019 and Viviani (2002).

In the sensors of the present invention, R¹ can be any suitable bioluminescent protein. One very well-known example is the class of proteins known as luciferases which catalyse an energy-yielding chemical reaction in which a specific biochemical substance, a luciferin (a naturally occurring fluorophore), is oxidized by an enzyme having a luciferase activity (Hastings, 1996). A great diversity of organisms, both prokaryotic and eukaryotic, including species of bacteria, algae, fungi, insects, fish and other marine forms can emit light energy in this manner and each has specific luciferase activities and luciferins which are chemically distinct from those of other organisms. Luciferin/luciferase systems are very diverse in form, chemistry and function. Bioluminescent proteins with luciferase activity are thus available from a variety of sources or by a variety of means. Examples of bioluminescent proteins with luciferase activity may be found in U.S. Pat. Nos. 5,229,285, 5,219,737, 5,843,746, 5,196,524, and 5,670,356. Two of the most widely used luciferases are: (i) Renilla luciferase (from R. reniformis), a 35 kDa protein, which uses coelenterazine as a substrate and emits light at 480 nm (Lorenz et al., 1991); and (ii) Firefly luciferase (from Photinus pyralis), a 61 kDa protein, which uses luciferin as a substrate and emits light at 560 nm (de Wet et al., 1987).

Gaussia luciferase (from Gaussia princeps) has been used in biochemical assays (Verhaegen et al., 2002). Gaussia luciferase is a 20 kDa protein that oxidises coelenterazine in a rapid reaction resulting in a bright light emission at 470 nm.

Luciferases useful for the present invention have also been characterized from Anachnocampa sp (WO 2007/019634). These enzymes are about 59 kDa in size and are ATP-dependent luciferases that catalyse luminescence reactions with emission spectra within the blue portion of the spectrum.

Biologically active variants or fragments of naturally occurring bioluminescent protein can readily be produced by those skilled in the art. Three examples of such variants useful for the invention are RLuc2 (Loening et al., 2006), RLuc8 (Loening et al., 2006) and RLuc8.6-535 (Loening et al., 2007) which are each variants of Renilla luciferase. RLuc8 contains the mutations A55T, C124A, 5130A, K136R, A143M, M185V, M253L, and S287L relative to RLuc. RLuc2 contains the mutations M185V and Q235A relative to RLuc. A further example is NanoLuc™ (Hall et al., 2012). In a further preferred embodiment, the sequence of the BRET chemiluminescent donor is chosen to have greater thermal stability than sensor molecules incorporating native Renilla luciferase sensors. RLuc2 or RLuc8 are convenient examples of suitable choices, which consequently exhibit ≥5× or ≥10× higher luminance than sensors incorporating the native Renilla luciferase sequence. Such enhanced luminance has significant benefits as it permits more economical use of reagents for any given time resolution. Non-limiting examples of bioluminescent proteins are provided in Table 2.

TABLE 2
Exemplary bioluminescent proteins.

| | | | MW | Emission | Example of |
|---|---|---|---|---|---|
| Species | Name | Organism | kDa × 10⁻³ | (nm) | Substrate |
| Insect | FFluc | Photinus pyralis | ~61 | 560 | D-(−)-2-(6′- |
| | | (North American | | | hydroxybenzothiazolyl)- |
| | | Firefly) | | | D²-thiazoline-4- |
| | | | | | carboxylic acid, |
| | | | | | HBTTCA |
| | | | | | (C₁₁H₈N₂O₃S₂) |
| | | | | | (luciferin) |
| Insect | FF′luc | Luciola cruciata | | 560-590 | Luciferin |
| | | (Japanese Firefly) | | (many | |
| | | | | mutants) | |
| Insect | | Phengodid beetles | | | |
| | | (railroad worms) | | | |
| Insect | | Arachnocampa spp. | | | Luciferin |
| Insect | | Orphelia fultoni | | | |
| | | (North American | | | |
| | | glow worm) | | | |
| Insect | Clluc | Pyrophorus | | 546, 560, | Luciferin |
| | | plagiophthalamus | | 578 and 593 | |
| | | (click beetle) | | | |
| Jellyfish | Aequorin | Aequorea | 44.9 | 460-470 | Coelenterazine |
| Sea pansy | RLuc | Renilla reniformis | 36 | 480 | Coelenterazine |
| Sea pansy | RLuc8 | Renilla reniformis | 36 | 487 | Coelenterazine/ |
| (modified) | | (modified) | | (peak) | Deep Blue C |
| Sea pansy | RLuc2 | Renilla reniformis | 36 | 480 | Coelenterazine |
| (modified) | | (modified | | | |
| | | M185V/Q235A) | | | |
| Sea pansy | RLuc8.6-535 | Renilla reniformis | 36 | 535 | Coelenterazine |
| (modified) | | (modified) | | | |
| Sea pansy | Rmluc | Renilla mullerei | 36.1 | ~480 | Coelenterazine |
| Sea pansy | | Renilla kollikeri | | | |
| Crustacea | Vluc | Vargula | ~62 | ~460 | Coelenterazine |
| (shrimp) | | hilgendorfii | | | |
| Crustaeca | CLuc | Cypridina | 75 | 465 | Coelenterazine/ |
| | | (sea firefly) | | | Cypridina |
| | | | | | luciferin |
| Dinofagellate | | Gonyaulax | 130 | ~475 | Tetrapyrrole |
| (marine alga) | | polyedra | | | |
| Mollusc | | Latia | 170 | 500 | Enol formate, |
| | | (fresh water limpet) | | | terpene, |
| | | | | | aldehyde |
| Hydroid | | Obelia | ~20 | ~470 | Coelenterazine |
| | | biscuspidata | | | |
| Shrimp | | Oplophorus | 31 | 462 | Coelenterazine |
| | | gracilorostris | | | |
| Shrimp | | Oplophorus | 19 | ~460 | Furimazine |
| | | gracilorostris | | | |
| | | (NanoLuc) | | | |
| Others | Ptluc | Ptilosarcus | | ~490 | Coelenterazine |
| | Gluc | Gaussia | ~20 | ~475 | Coelenterazine |
| | Plluc | Pleuromamma | 22.6 | ~475 | Coelenterazine |

Alternative, non-luciferase, bioluminescent proteins that can be employed in this invention are any enzymes which can act on suitable substrates to generate a luminescent signal. Specific examples of such enzymes are β-galactosidase, lactamase, horseradish peroxidase, alkaline phosphatase, β-glucuronidase and β-glucosidase. Synthetic luminescent substrates for these enzymes are well known in the art and are commercially available from companies, such as Tropix Inc. (Bedford, Mass., USA).

An example of a peroxidase useful for the present invention is described by Hushpulian et al. (2007).

In some embodiments, R¹ can include, but is not limited to, a luciferase, a β-galactosidase, a lactamase, a horseradish peroxidase, an alkaline phosphatase, a β-glucuronidase and a β-glucosidase or a biologically active fragment or variant thereof.

In preferred embodiments, the bioluminescent protein is a luciferase. In some embodiments, R¹ is a luciferase selected from the group consisting of a Renilla luciferase, a Firefly luciferase, a Coelenterate luciferase, a North American glow worm luciferase, a click beetle luciferase, a railroad worm luciferase, a bacterial luciferase, a Gaussia luciferase, Aequorin, an Arachnocampa luciferase, or a biologically active variant or fragment of any one, or chimera of two or more, thereof. In some embodiments, R¹ comprises RLuc (SEQ ID NO: 49) or a biologically active fragment or variant thereof. In some embodiments, R¹ comprises RLuc8 (SEQ ID NO: 50) or a biologically active fragment or variant thereof. In some embodiments, R¹ comprises RLuc2 (SEQ ID NO: 51) or a biologically active fragment or variant thereof. In some embodiments, R¹ has an amino acid sequence which is at least 30%, 35%, 40%, 45%, 50%, 55%, 60%, 65%, 70%, 75%, 80%, 85%, 90%, 95%, 96%, 97%, 98%, 99% or 100% identical to the sequence provided in any one or more of SEQ ID NO: 49, SEQ ID NO: 50 and SEQ ID NO: 51. In some embodiments, R¹ has an amino acid sequence which is at least 30%, 35%, 40%, 45%, 50%, 55%, 60%, 65%, 70%, 75%, 80%, 85%, 90%, 95%, 96%, 97%, 98%, 99% or 100% identical to the sequence provided in any one or more of SEQ ID NO: 1, SEQ ID NO: 2, SEQ ID NO: 3; SEQ ID NO: 4, SEQ ID NO: 32 and SEQ ID NO: 33.

As used herein, a “biologically active fragment” is a portion of a polypeptide as described herein which maintains a defined activity of the full-length polypeptide. For example, in embodiments where the full-length polypeptide is a bioluminescent protein, the “biologically active fragment” maintains at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99% or 100% of the activity of the full-length bioluminescent protein, wherein activity is a measure of the ability of the polypeptide to convert a substrate into an activated product which then releases energy as it relaxes.

Biologically active fragments are typically at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99% identical to the naturally occurring and/or defined polypeptide. As used herein, a “biologically active variant” is a sequence variant of a polypeptide as described herein which maintains a defined activity of the native polypeptide. Biologically active variants are typically at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99% identical to the naturally occurring and/or defined polypeptide. As used herein, a “biologically active variant” includes a fusion protein. The fusion protein comprises the bioluminescent protein (or a fragment or variant thereof) fused to a protein, polypeptide or peptide. The protein, polypeptide or peptide can be a tag, for example a solubility tag or a purification tag. The fusion protein may optionally comprise an amino acid sequence that permits cleavage of the bioluminescent protein (or a fragment or variant thereof) from the protein, polypeptide or peptide.

In some embodiments, R¹ is a biologically active variant of a bioluminescent protein comprising at least one less cysteine residue when compared to the corresponding naturally occurring protein. For example, the biologically active variant may comprise at least one less cysteine residue, at least two less cysteine residues or at least three less cysteine residues when compared to the corresponding naturally occurring protein. The cysteine residue may be replaced with a naturally or non-naturally occurring amino acid. In some embodiments, the cysteine is replaced by a serine, valine, alanine, threonine or selenocysteine. In some embodiments, the variant bioluminescent protein lacks a cysteine residue at a position corresponding to amino acid position 24, at a position corresponding to amino acid position 73 or at a position corresponding to amino acid position 124 of RLuc (SEQ ID NO: 49). In some embodiments, the variant bioluminescent protein lacks a cysteine residue at positions corresponding to amino acid positions 24 and 73, at positions corresponding to amino acid positions 24 and 124 or at positions corresponding to amino acid positions 73 and 124 of RLuc (SEQ ID NO: 49). In some embodiments, the variant bioluminescent protein lacks a cysteine residue at positions corresponding to amino acid positions 24, 73 and 124 of RLuc (SEQ ID NO: 49). In some embodiments, the variant bioluminescent protein lacks a cysteine residue at a position corresponding to amino acid position 24 of RLuc8 (SEQ ID NO: 50). In some embodiments, the variant bioluminescent protein lacks a cysteine residue at a position corresponding to amino acid position 73 of RLuc8 (SEQ ID NO: 50). In some embodiments, the variant bioluminescent protein lacks a cysteine residue at a position corresponding to amino acid positions 24 and 73 of RLuc8. In some embodiments, the variant bioluminescent protein comprising a polypeptide sequence selected from the group consisting of SEQ ID NO: 8, SEQ ID NO: 9 and SEQ ID NO: 10. As used herein, the phrase “at a position corresponding to amino acid position” or variations thereof refers to the relative position of the amino acid compared to surrounding amino acids. In this regard, in some embodiments a polypeptide of the invention may have deletional or substitutional mutation which alters the relative positioning of the amino acid when aligned against, for instance, SEQ ID NO: 49.

In some embodiments, R¹ is a biologically active variant of a bioluminescent protein comprising at least one cysteine residue when the corresponding naturally occurring protein does not comprise a cysteine residue at the same sequence location. In some embodiments, R¹ is a biologically active variant of a bioluminescent protein comprising at least one exposed cysteine residue when the corresponding naturally occurring protein does not comprise an exposed cysteine residue. As used herein, an “exposed cysteine” is one which is located near to or on the surface of the protein such that the side-chain of the cysteine is available to react with L or R² to form the sensor molecule described herein. The mutated cysteine residue achieved through an amino acid change to a cysteine or through the provision of an exposed cysteine residue can act as, or form part of, the linking element in the sensors defined herein. For example, the side-chain of the mutated cysteine can react with a thiol reactive group in R² to form a sensor as defined herein. In some embodiments, R¹ is a biologically active variant of a bioluminescent protein comprising at least one cysteine residue when the corresponding naturally occurring protein does not comprise a cysteine residue at the same sequence location and at least one less cysteine residue when the corresponding naturally occurring protein does comprises a cysteine residue at the same sequence location.

In a preferred embodiment, a bioluminescent protein with a small molecular weight is used to prevent or reduce an inhibition of the interaction between the hydrolase with the sensor due to steric hindrance. The bioluminescent protein preferably comprises a single polypeptide chain. Also the bioluminescent proteins preferably do not form oligomers or aggregates. The bioluminescent proteins Renilla luciferase, Gaussia luciferase and Firefly luciferase meet all or most of these criteria.

In some embodiments, the bioluminescent protein is capable of modifying a substrate. As used herein, the term “substrate” refers to any molecule that can be used in conjunction with a chemiluminescent donor to generate or absorb luminescence. The choice of the substrate can impact on the wavelength and the intensity of the light generated by the chemiluminescent donor. In some embodiments, the bioluminescent protein has a substrate selected from luciferin, calcium, coelenterazine, a derivative or analogue of coelenterazine or a derivative or analogue of luciferin. In preferred embodiments, the substrate is luciferin, calcium, coelenterazine, or a derivative or analogue of coelenterazine.

Coelenterazine is a widely known substrate which occurs in cnidarians, copepods, chaetognaths, ctenophores, decapod shrimps, mysid shrimps, radiolarians and some fish taxa (Greer and Szalay, 2002). For Renilla luciferase for example, coelenterazine analogues/derivatives are available that result in light emission between 418 and 547 nm (Inouye et al., 1997, Loening et al., 2007). A coelenterazine analogue/derivative (400A, DeepBlueC) has been described emitting light at 400 nm with Renilla luciferase (WO 01/46691). Other examples of coelenterazine analogues/derivatives are EnduRen, Prolume purple, Prolume purple II, Prolume purple III, ViviRen and Furimazine. Other examples of coelenterazine analogues/derivatives include, but are not limited to, compounds disclosed in WO/2014/036482 and US20140302539.

As used herein, the term “luciferin” is defined broadly and refers to a class of light-emitting biological pigments found in organisms capable of bioluminescence as well as synthetic analogues or functionally equivalent chemicals, which are oxidised in the presence of the enzyme luciferase to produce oxyluciferin and energy in the form of light. D-luciferin, or 2-(6-hydroxybenzothiazol-2-yl)-2-thiazoline-4-carboxylic acid, was first isolated from the firefly Photinus pyralis. Since then, various chemically distinct forms of luciferin have been discovered and studied from various different organisms, mainly from the ocean, for example fish and squid, however, many have been identified in land dwelling organisms, for example, worms, beetles and various other insects (Day et al., 2004; Viviani, 2002). As used herein, luciferin also includes derivatives or analogues of luciferin.

In addition to entirely synthetic luciferin, such as cyclic alkylaminoluciferin (CycLuc1), there are at least five general types of biologically evolved luciferin, which are each chemically different and catalysed by chemically and structurally different luciferases that employ a wide range of different cofactors. First, is firefly luciferin, the substrate of firefly luciferase, which requires ATP for catalysis (EC 1.13.12.7). Second, is bacterial luciferin, also found in some squid and fish, which consists of a long chain aldehyde and a reduced riboflavin phosphate. Bacterial luciferase is FMNH-dependent. Third, is dinoflagellate luciferin, a tetrapyrrolic chlorophyll derivative found in dinoflagellates (marine plankton), the organisms responsible for night-time ocean phosphorescence. Dinoflagellate luciferase catalyses the oxidation of dinoflagellate luciferin and consists of three identical and catalytically active domains. Fourth, is the imidazolopyrazine vargulin, which is found in certain ostracods and deep-sea fish, for example, Porichthys. Last, is coelenterazine (an imidazolpyrazine), the light-emitter of the protein aequorin, found in radiolarians, ctenophores, cnidarians, squid, copepods, chaetognaths, fish and shrimp.

In some embodiments, the bioluminescent protein requires a co-factor. Examples of co-factors include, but are not necessarily limited to, ATP, magnesium, oxygen, FMNH₂, calcium, or a combination of any two or more thereof.

Hydrolases

A hydrolase is an enzyme which catalyses a hydrolysis reaction. Hydrolysis is the cleavage of a chemical bond by the addition of water. Hydrogen is added to one side of the broken chemical bond and a hydroxyl is added to the other side of the broken chemical bond. For example:

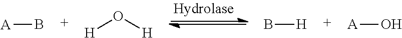

As used herein, the term “hydrolase” refers to any protein capable of catalysing a hydrolysis reaction. Hydrolases are classified as EC 3 in the EC number (Enzyme commission number) classification of enzymes. They can be further classified into subclasses based on the chemical bond they hydrolyse. In some embodiments, the hydrolase can be a polypeptide with an EC number selected from the following group consisting of EC 3.1; EC 3.2; EC 3.3; EC 3.4; EC 3.5; EC 3.6; EC 3.7; EC 3.8; EC 3.9; EC 3.10; EC 3.11 and EC 3.13, or a fragment or variant of any of the aforementioned.

Details of polypeptides defined by the above EC numbers are as described in Enzyme Nomenclature 1992 [Academic Press, San Diego, Calif., ISBN 0-12-227164-5 (hardback), 0-12-227165-3 (paperback)] with Supplement 1 (1993), Supplement 2 (1994), Supplement 3 (1995), Supplement 4 (1997) and Supplement 5 (in Tipton 1994; Barrett 1995; Barrett 1995; Barrett 1997, and Nomenclature Committee 1999, respectively). Details are also available from the Nomenclature Committee of the International Union of Biochemistry and Molecular Biology (http://www.chem.gmul.ac.uk/iubmb/enzyme/EC3/), and ExPASy http://enzyme.expasy.org/EC/3.-.-.-), amongst others.

The amino acid sequences of the relevant polypeptides can be readily obtained by a person skilled in the art. For example, the sequences are available via ExPASy http://enzyme.expasy.org/EC/3.-.-.-.

Exemplary hydrolases include, but are not limited to, hydrolases that act on ester bonds, hydrolases that act on ether bonds, hydrolases that act on peptide bonds, hydrolases that act on carbon-nitrogen bonds other than peptide bonds, hydrolases that act on acid anhydrides, hydrolases that act on carbon-carbon bonds, hydrolases that act on halide bonds, hydrolases that act on phosphorous-nitrogen bonds, hydrolases that act on sulphur-nitrogen bonds, hydrolases that act on carbon-phosphorous bonds, hydrolases that act on carbon-sulphur bonds, hydrolases that act on sulphur-sulphur bonds and glycosylases. In some examples, the hydrolases that act on ester bonds include carboxylesterase, arylesterase, acetylesterases, acetylcholinesterase, cholinesterase, thioesterases (such as acetyl-CoA hydrolase, glutathione thiolesterase), phosphatases (alkaline phosphatase), sulfuric ester hydrolase and lipases. In some examples, the hydrolases that act on peptide bonds include serine and cysteine proteases, carboxy- and aminopeptidases, metallopeptidases, dipeptidases, dipeptidyl-peptidases and tripeptidyl-peptidases. In some examples, hydrolases that act on carbon-nitrogen bonds other than peptide bonds include amidases which target linear amides or cyclic amides. In some embodiments, the hydrolase is a β-lactamase. In some examples, the hydrolase is a glycosidase, such as an α-glycosidase or a β-glycosidase. Glycosidases hydrolyse N-, O- and S-glycosyl compounds and include, but are not limited to, amylases, maltases, sucrases, lactases and galactosidases. In some examples, the hydrolase is a nucleoside hydrolase such as a purine nucleosidease or a pyrimidine nucleosidease. In some examples, the hydrolase is a nucleotide hydrolase such as GTPase. In some examples, the hydrolase is an exonuclease, endonuclease or a DNA glycosylase. In some examples, the hydrolase is a dealkylase. In some examples, the hydrolase is a dehalogenase.

In some embodiments, the hydrolase is selected from the group consisting of cholinesterase, esterase, lipase, protease, phosphatase, nuclease, glycosidase, DNA glycosylases and acid anhydride hydrolase. In some examples, the hydrolase is selected from the group consisting of cholinesterase, lipase, protease and phosphatase. In some embodiments, the hydrolase is an esterase. One example of a suitable hydrolase is porcine liver esterase. In some embodiments, the hydrolase is a phosphatase.

R¹ and R²

Any number of R¹ and R² combinations can be used in the sensors of the present invention. A person skilled in the art would be able to select an R¹ and R² pair which permits efficient energy transfer. In preferred embodiments, the separation and relative orientation of R¹ and R² is within ±50% of the Förster distance. As used herein, the term “the separation and relative orientation of R¹ and R² is within ±50% of the Förster distance” refers to the steady state RET measurements which can be carried out within a range of ±50% of R₀ (Förster 1948; Förster 1960). This phrase encompasses an efficiency of luminescence energy transfer from the chemiluminescent donor domain to the acceptor domain in the range of 10-90%. In some embodiments, the Förster distance of the chemiluminescent donor domain and the acceptor domain is at least 4 nm, is at least 4.5 nm, is at least 5.0 nm, is at least 5.6 nm, or is at least 6 nm. In some embodiments, the Förster distance of the chemiluminescent donor domain and the acceptor domain is between about 4 nm and about 10 nm, is between about 4.5 nm and about 10 nm, is between about 5.0 nm and about 10 nm, is between about 5.6 nm and about 10 nm or is between about 6 nm and about 10 nm.

A criterion which should be considered in determining suitable pairings is the relative emission/fluorescence spectrum of the acceptor molecule (R²) compared to that of the bioluminescent protein (R¹). In some embodiments, the emission spectrum of the bioluminescent protein should overlap with the absorbance spectrum of the acceptor molecule such that the light energy from the donor luminescence emission is at a wavelength that is able to excite the acceptor molecule and thereby promote acceptor molecule fluorescence when the two molecules are in a proper proximity and orientation with respect to one another. To study a potential pairing, fusions (for example) are prepared containing the selected bioluminescent protein and acceptor domain without the blocking group B and are tested (see Examples).

It should also be confirmed that the donor and acceptor molecule do not spuriously associate with each other.

The donor emission can be manipulated by modifications to the substrate. In the case of Renilla luciferases the substrate is coelenterazine. The rationale behind altering the donor emission is to improve the resolution between donor emission and acceptor emissions. The original BRET system uses the Renilla luciferase as donor, EYFP (or Topaz) as the acceptor and coelenterazine h derivative as the substrate. These components when combined in a BRET assay, generate light in the 475-480 nm range for the bioluminescent protein and the 525-530 nm range for the acceptor molecule, giving a spectral resolution of 45-55 nm.

Renilla luciferase generates a broad emission peak overlapping substantially the GFP emission, which in turn contributes to decrease the signal to noise of the system. One BRET system for use in the present invention has coel400a as the Renilla luciferase substrate and provides broad spectral resolution between donor and acceptor emission wavelengths (˜105 nm).

Various coelenterazine derivatives are known in the art, including coel400a, that generate light at various wavelengths (distinct from that generated by the wild type coelenterazine) as a result of Renilla luciferase activity. A person skilled in the art would appreciate that because the light emission peak of the donor has changed, it is necessary to select an acceptor molecule which will absorb light at this wavelength and thereby permit efficient energy transfer. Spectral overlapping between light emission of the donor and the light absorption peak of the acceptor is one condition among others for an efficient energy transfer.

Examples of further bioluminescent protein and acceptor molecule pairs are provided in Table 3.

TABLE 3
Exemplary BRET bioluminescent protein (R¹) and acceptor molecule (R²) pairs.

| | | Substrate | | Wavelength |
|---|---|---|---|---|
| Bioluminescent | | wavelength | Acceptor | of acceptor |
| protein (R¹) | Substrate | (peak) | molecule (R²) | (Ex/Em) |
| RLuc2 | Native | 470 nm | Fluorescein | 495/519 nm |
| RLuc8 | Coelenterazine | | | |
| RLuc2 | Native | 470 nm | Acridine yellow | 470/550 nm |
| RLuc8 | Coelenterazine | | | |
| RLuc2 | Native | 470 nm | Nile red | 485/525 nm |
| RLuc8 | Coelenterazine | | | |
| RLuc2 | Native | 470 nm | Red 613 | 480/613 nm |
| RLuc8 | Coelenterazine | | | |
| RLuc2 | Native | 470 nm | TruRed | 490/695 nm |
| RLuc8 | Coelenterazine | | | |
| RLuc | Coelenterazine | 470 nm | Fluorescein | 490/525 nm |
| RLuc2 | h | | | |
| RLuc8 | | | | |
| RLuc | Coelenterazine | 470 nm | Acridine yellow | 470/550 nm |
| RLuc2 | h | | | |
| RLuc8 | | | | |
| RLuc | Coelenterazine | 470 nm | Nile red | 485/525 nm |
| RLuc2 | h | | | |
| RLuc8 | | | | |
| RLuc | Coelenterazine | 470 nm | Red 613 | 480/613 nm |
| RLuc2 | h | | | |
| RLuc8 | | | | |
| RLuc | Coelenterazine | 470 nm | TruRed | 490/695 nm |
| RLuc2 | h | | | |
| RLuc8 | | | | |
| RLuc | Coelenterazine | 400 nm | Quin-2 | 365/490 nm |
| RLuc2 | 400a | | | |
| RLuc8 | | | | |
| RLuc | Coelenterazine | 400 nm | Pacific blue | 403/551 nm |
| RLuc2 | 400a | | | |
| RLuc8 | | | | |
| RLuc | Coelenterazine | 400 nm | Dansyl chloride | 380/475 nm |
| RLuc2 | 400 | | | |
| RLuc8 | | | | |
| Firefly | Luciferin | 560 nm | Cyanine Cy3 | 575/605 nm |
| luciferase | | | | |
| Firefly | Luciferin | 560 nm | Texas red | 590/615 nm |
| luciferase | | | | |
| FFLuc | Luciferin | 560 nm | AF680 | 679/702 nm |
| PpyRE8 | | | | |
| PpyRE10 | | | | |
| FFLuc | Luciferin | 560 nm | AF750 | 749/775 nm |
| PpyRE8 | | | | |
| PpyRE10 | | | | |
| NanoLuc | Furimazine | 460 nm | Fluorescein | 495/519 nm |
| NanoLuc | Furimazine | 460 nm | Acridine yellow | 470/550 nm |
| NanoLuc | Furimazine | 460 nm | Nile red | 485/525 nm |
| NanoLuc | Furimazine | 460 nm | Red 613 | 480/613 nm |
| NanoLuc | Furimazine | 460 nm | TruRed | 490/695 nm |
| NanoLuc | Furimazine | 460 nm | Oregon Green | 496/516 nm |
| NanoLuc | Furimazine | 460 nm | diAcFAM | 494/526 nm |
| NanoLuc | Furimazine | 460 nm | AlexFluor488 | 494/517 nm |
| NanoLuc | Furimazine | 460 nm | TMR | 555/585 nm |
| NanoLuc | Furimazine | 460 nm | Halotag NCT | 595/635 nm |
| NanoLuc | Furimazine | 460 nm | HalotagBRET | 525/618 nm |
| | | | 618 | |
| NanoLuc | Native | 460 nm | Fluorescein | 495/519 nm |
| | Coelenterazine | | | |
| NanoLuc | Native | 460 nm | Acridine yellow | 470/550 nm |
| | Coelenterazine | | | |
| NanoLuc | Native | 460 nm | Nile red | 485/525 nm |
| | Coelenterazine | | | |
| NanoLuc | Native | 460 nm | Red 613 | 480/613 nm |
| | Coelenterazine | | | |
| NanoLuc | Native | 460 nm | TruRed | 490/695 nm |
| | Coelenterazine | | | |
| NanoLuc | Native | 460 nm | Oregon Green | 496/516 nm |
| | Coelenterazine | | | |
| NanoLuc | Native | 460 nm | diAcFAM | 494/526 nm |
| | Coelenterazine | | | |
| NanoLuc | Native | 460 nm | AlexFluor488 | 494/517 nm |
| | Coelenterazine | | | |
| NanoLuc | Native | 460 nm | TMR | 555/585 nm |
| | Coelenterazine | | | |
| NanoLuc | Native | 460 nm | Halotag NCT | 595/635 nm |
| | Coelenterazine | | | |
| NanoLuc | Native | 460 nm | HalotagBRET | 525/618 |
| | Coelenterazine | | 618 | |
| NanoLuc | Coelenterazine | 460 nm | Fluorescein | 495/519 nm |
| | h | | | |
| NanoLuc | Coelenterazine | 460 nm | Acridine yellow | 470/550 nm |
| | h | | | |
| NanoLuc | Coelenterazine | 460 nm | Nile red | 485/525 nm |
| | h | | | |
| NanoLuc | Coelenterazine | 460 nm | Red 613 | 480/613 nm |
| | h | | | |
| NanoLuc | Coelenterazine | 460 nm | TruRed | 490/695 nm |
| | h | | | |
| NanoLuc | Coelenterazine | 460 nm | Oregon Green | 496/516 nm |
| | h | | | |
| NanoLuc | Coelenterazine | 460 nm | diAcFAM | 494/526 nm |
| | h | | | |
| NanoLuc | Coelenterazine | 460 nm | AlexFluor488 | 494/517 nm |
| | h | | | |
| NanoLuc | Coelenterazine | 460 nm | TMR | 555/585 nm |
| | h | | | |
| NanoLuc | Coelenterazine | 460 nm | Halotag NCT | 595/635 nm |
| | h | | | |
| NanoLuc | Coelenterazine | 460 nm | HalotagBRET | 525/618 |
| | h | | 618 | |
| RLuc | Prolume Purple | 405 nm | Quin-2 | 365/490 nm |
| RLuc2 | Substrate | | | |
| RLuc8 | | | | |
| RLuc | Prolume Purple | 405 nm | Pacific blue | 403/551 nm |
| RLuc2 | Substrate | | | |
| RLuc8 | | | | |
| RLuc | Prolume Purple | 405 nm | Dansyl chloride | 380/475 nm |
| RLuc2 | Substrate | | | |
| RLuc8 | | | | |
| RLuc | Prolume Purple | 400 nm | Quin-2 | 365/490 nm |
| RLuc2 | Substrate II | | | |
| RLuc8 | | | | |
| RLuc | Prolume Purple | 400 nm | Pacific blue | 403/551 nm |
| RLuc2 | Substrate II | | | |
| RLuc8 | | | | |
| RLuc | Prolume Purple | 400 nm | Dansyl chloride | 380/475 nm |
| RLuc2 | Substrate II | | | |
| RLuc8 | | | | |
| RLuc | Prolume Purple | 410 nm | Quin-2 | 365/490 nm |
| RLuc2 | Substrate III | | | |
| RLuc8 | | | | |
| RLuc | Prolume Purple | 410 nm | Pacific blue | 403/551 nm |
| RLuc2 | Substrate III | | | |
| RLuc8 | | | | |
| RLuc | Prolume Purple | 410 nm | Dansyl chloride | 380/475 nm |
| RLuc2 | Substrate III | | | |
| RLuc8 | | | | |

Bioluminescent Resonance Energy Transfer (BRET)

As used herein, “BRET” or “bioluminescent resonance energy transfer” is a proximity assay based on the non-radioactive transfer of energy between the bioluminescent protein donor and the acceptor molecule. “Bioluminescent resonance energy transfer” and “BRET” are used interchangeably.

Cleavage of the hydrolysable bond of the sensor described herein by a hydrolase produces a change in BRET ratio. Energy transfer occurring between the bioluminescent protein and acceptor molecule is presented as calculated ratios from the emissions measured using optical filters (one for the acceptor molecule emission and the other for the bioluminescent protein emission) that select specific wavelengths (see equation 1).

Eₐ/E_{d}=BRET ratio (1)

where Eₐ is defined as the acceptor molecule emission intensity (emission light is selected using a specific filter adapted for the emission of the acceptor) and E_{d} is defined as the bioluminescent protein emission intensity (emission light is selected using a specific filter adapted for the emission of the bioluminescent protein).

It should be readily appreciated by those skilled in the art that the optical filters may be any type of filter that permits wavelength discrimination suitable for BRET. For example, optical filters used in accordance with the present invention can be interference filters, long pass filters, short pass filters, etc. Intensities (usually in counts per second (CPS) or relative luminescence units (RLU)) of the wavelengths passing through filters can be quantified using either a solid state micro-photomultiplier (micro-PMT), photo-multiplier tube (PMT), photodiode, including a cascade photodiode, photodiode array or a sensitive camera such as a charge coupled device (CCD) camera. The quantified signals are subsequently used to calculate BRET ratios and represent energy transfer efficiency. The BRET ratio increases with increasing intensity of the acceptor emission.

Generally, a ratio of the acceptor emission intensity over the donor emission intensity is determined (see equation 1), which is a number expressed in arbitrary units that reflects energy transfer efficiency. The ratio increases with an increase of energy transfer efficiency (see Xu et al., 1999).

Energy transfer efficiencies can also be represented using the inverse ratio of donor emission intensity over acceptor emission intensity (see equation 2). In this case, ratios decrease with increasing energy transfer efficiency. Prior to performing this calculation the emission intensities are corrected for the presence of background light and auto-luminescence of the substrate. This correction is generally made by subtracting the emission intensity, measured at the appropriate wavelength, from a control sample containing the substrate but no bioluminescent protein, acceptor molecule, sensor or polypeptide of the invention.

E_{d}/Eₐ=BRET ratio (2)

where Eₐ and E_{d} are as defined above.

The light intensity of the bioluminescent protein and acceptor molecule emission can also be quantified using a monochromator-based instrument such as a spectrofluorometer, a charged coupled device (CCD) camera or a diode array detector. Using a spectrofluorometer, the emission scan is performed such that both bioluminescent protein and acceptor molecule emission peaks are detected upon addition of the substrate. The areas under the peaks or the intensities at λₘₐₓ or at wavelengths defined by any arbitrary intensity percentage relative to the maximum intensity can be used to represent the relative light intensities and may be used to calculate the ratios, as outlined above. Any instrument capable of measuring lights for the bioluminescent protein and acceptor molecule from the same sample can be used to monitor the BRET system of the present invention.

In an alternative embodiment, the acceptor molecule emission alone is suitable for effective detection and/or quantification of BRET. In this case, the energy transfer efficiency is represented using only the acceptor emission intensity. It would be readily apparent to one skilled in the art that in order to measure energy transfer, one can use the acceptor emission intensity without making any ratio calculation. This is due to the fact that ideally the acceptor molecule will emit light only if it absorbs the light transferred from the bioluminescent protein. In this case only one light filter is necessary.

In a related embodiment, the bioluminescent protein emission alone is suitable for effective detection and/or quantification of BRET. In this case, the energy transfer efficiency is calculated using only the bioluminescent protein emission intensity. It would be readily apparent to one skilled in the art that in order to measure energy transfer, one can use the donor emission intensity without making any ratio calculation. This is due to the fact that as the acceptor molecule absorbs the light transferred from the bioluminescent protein there is a corresponding decrease in detectable emission from the bioluminescent protein. In this case only one light filter is necessary.

In an alternative embodiment, the energy transfer efficiency is represented using a ratiometric measurement which only requires one optical filter for the measurement. In this case, light intensity for the donor or the acceptor is determined using the appropriate optical filter and another measurement of the samples is made without the use of any filter (intensity of the open spectrum). In this latter measurement, total light output (for all wavelengths) is quantified. Ratio calculations are then made using either equation 3 or 4. For the equation 3, only the optical filter for the acceptor is required. For the equation 4, only the optical filter for the donor is required.

Eₐ/E₀-Eₐ=BRET ratio or =Eₒ-Eₐ/Eₐ (3)

Eₒ-E_{d}/E_{d}=BRET ratio or =E_{d}/Eₒ-E_{d} (4)

where Eₐ and E_{d} are as defined above and Eₒ is defined as the emission intensity for all wavelengths combined (open spectrum).

It should be readily apparent to a person skilled in the art that further equations can be derived from equations 1 through 4. For example, one such derivative involves correcting for background light present at the emission wavelength for the bioluminescent protein and/or acceptor molecule.

In performing a BRET assay, light emissions can be determined from each well using the BRETCount. The BRETCount instrument is a modified TopCount, wherein the TopCount is a microtiterplate scintillation and luminescence counter sold by Packard Instrument (Meriden, Conn.). Unlike classical counters which utilise two photomultiplier tubes (PMTs) in coincidence to eliminate background noise, TopCount employs single-PMT technology and time-resolved pulse counting for noise reduction to allow counting in standard opaque microtiter plates. The use of opaque microtiterplates can reduce optical crosstalk to negligible level. TopCount comes in various formats, including 1, 2, 6 and 12 detectors (PMTs), which allow simultaneous reading of 1, 2, 6 or 12 samples, respectively. Beside the BRETCount, other commercially available instruments are capable of performing BRET: the Victor 2 (Wallac, Finland (Perkin Elmer Life Sciences)) and the Fusion (Packard Instrument, Meriden). BRET can be performed using readers that can detect at least the acceptor molecule emission and preferably two wavelengths (for the acceptor molecule and the bioluminescent protein) or more.

As the person skilled in the art would understand, BRET requires that the sensor comprise a chemiluminescent donor domain (in this case a bioluminescent protein) and an acceptor domain. In some embodiments, the spatial location and/or dipole orientation of the chemiluminescent donor domain relative to the acceptor domain is altered when the hydrolysable bond is cleaved by a hydrolase resulting in a change in the BRET ratio. As used herein, the term “spatial location” refers to the three dimensional positioning of the donor relative to the acceptor molecule which changes as a result of the protease cleaving the sensor molecule, such that the donor domain is no longer linked to the acceptor domain via the target sequence. As used herein, the term “dipole orientation” refers to the direction in three-dimensional space of the dipole moment associated either with the donor and/or the acceptor molecule relative their orientation in three-dimensional space. The dipole moment is a consequence of a variation in electrical charge over a molecule.

In some embodiments, cleavage of the hydrolysable bond by a hydrolase results in a change in absorption and/or emission spectra for the fluorescent acceptor domain, R². For example, in some embodiments, cleavage of the hydrolysable bond is cleaved by a hydrolase resulting in a change in maximal excitation (Ex) and/or emission (Em) wavelengths for the fluorescent acceptor domain. These changes can result in a change in the BRET ratio.

Cleavage of the hydrolysable bond by a hydrolase results in a change in BRET ratio, for example, cleavage of the hydrolysable bond by a hydrolase can result in a change in BRET ratio between about 2% to about 100% of the maximum observed BRET ratio. As used herein, “the maximum observed BRET ratio” is the BRET ratio observed for R¹-L-R² or R²-L-R¹ (that is for R² joined to R¹ via an optional linking element in the absence of B). In some embodiments, the change in BRET ratio is between about 5% to about 95%, about 15% to about 50%, or about 15% to about 40%, of the maximum observed BRET ratio. In some embodiments, cleavage of the hydrolysable bond by a hydrolase results in a change in BRET ratio which is ≥2% of the maximum observed BRET ratio. In some embodiments, cleavage of the hydrolysable bond by a hydrolase results in a change in BRET ratio which is ≥5%, ≥10%, ≥20%, ≥30%, ≥40%, ≥50%, ≥60%, ≥70%, ≥80%, ≥90% or ≥95% of the maximum observed BRET ratio. A change in the BRET ratio of 15% or more increases the signal to noise ratio of hydrolase detection. This results in a superior limit of detection for any given sampling time and more precise measurement of the concentration of hydrolase. Alternatively, at a fixed limit of detection, the greater change in BRET ratio facilitates shorter signal integration times and therefore more rapid detection.

In some embodiments, cleavage of the hydrolysable bond by a hydrolase can result in a change in BRET ratio by greater than about 2 fold, by greater than about 3 fold, by greater than about 4 fold, by greater than about 5 fold, by greater than about 10 fold, by greater than about 20 fold, by greater than about 30 fold, by greater than about 40 fold, by greater than about 50 fold, by greater than about 60 fold, by greater than about 70 fold, by greater than about 80 fold, by greater than about 90 fold, or by greater than about 100 fold. In some embodiments, the change in BRET ratio is between about 1 fold to about 60 fold, between about 2 fold to about 50 fold, between about 3 fold to about 40 fold, or between about 4 fold to about 30 fold.

As used herein, “Stokes shift” is the difference in wavelength between positions of the band maxima of the absorption and emission spectra of the same electronic transition. Preferably, the acceptor domain has a large Stokes shift. A large Stokes shift is desirable because a large difference between the positions of the band maxima of the absorption and emission spectra makes it easier to eliminate the reflected excitation radiation from the emitted signal. In some embodiments, the acceptor domain has a Stokes shift of greater than about 50 nm. In some embodiments, the acceptor domain has a Stokes shift of between about 50 nm and about 350 nm, between about 50 nm and about 150 nm. In some embodiments, the acceptor domain has a Stokes shift of greater than about 90 nm, for example 100 nm, 110 nm, 120 nm, 130 nm, 140 nm or 150 nm.

Composition and Kits

The sensors of the present invention may be included in compositions for use in detecting hydrolases. In some embodiments, the sensors described herein may be included in compositions for detecting an esterase. For example, in some embodiments, the sensors described herein may be included in compositions for detecting a cholinesterase or a lipase. In other embodiments, the sensors described herein may be included in compositions for detecting a phosphatase. For example, in some embodiments, the sensors described herein may be included in compositions for detecting alkaline phosphatase. In yet other embodiments, the sensors described herein may be included in compositions for detecting a glycosidase. For example, in some embodiments, the sensors described herein may be included in compositions for detecting lactase, glucosidase, galactosidase or maltase. In yet another embodiment, the sensors described herein may be included in compositions for detecting a protease. For example, in some embodiments, the sensors described herein may be included in compositions for detecting caspase. In yet another embodiment, the sensors described herein may be included in compositions for detecting a nuclease or a DNA/RNA hydrolase. For example, in some embodiments, the sensors described herein may be included in compositions for detecting ribonuclease or endonuclease. In yet another embodiment, the sensors described herein may be included in compositions for detecting a β-lactamase.

In some embodiments, there is provided a composition comprising a sensor in accordance with the present invention and an acceptable carrier. As used herein, the term “acceptable carrier” includes any and all solids or solvents (such as phosphate buffered saline buffers, water, saline) dispersion media, coatings, and the like, compatible with the methods and uses of the present invention. The acceptable carriers must be ‘acceptable’ in the sense of being compatible with the other ingredients of the composition and not inhibiting or damaging the hydrolases being tested for. Generally, suitable acceptable carriers are known in the art and are selected based on the end use application.

The sensors of the present invention can be included in kits for use in detecting hydrolases. In some embodiments, there is provided a kit comprising a sensor in accordance with the present invention and instructions for use. In one example, the kit comprises a sensor in accordance with the present invention, instructions for use and a substrate suitable for the bioluminescent protein of the sensor.

Methods and Uses

As the skilled person would appreciate, the sensors of the present invention can be used to detect the presence or absence of a hydrolase in a sample, and if present may also be used to determine the activity of the hydrolase (FIG. 10). Therefore, in one aspect there is provided a method of detecting a hydrolase in a sample, the method comprising (i) contacting a sample with a sensor molecule of the invention; and (ii) detecting a change in BRET ratio, wherein the change in the BRET ratio corresponds to the presence of a hydrolase in the sample (see, for example, FIG. 10A). For example, in some embodiments there is provided a method of detecting an esterase in a sample, the method comprising (i) contacting a sample with a sensor molecule of the invention; and (ii) detecting a change in BRET ratio, wherein the change in the BRET ratio corresponds to the presence of an esterase in the sample. As would be understood by a person skilled in the art, “contacting” in step (i) occurs under conditions that are suitable for hydrolysis of the sensor by the hydrolase. In some embodiments, the method comprises contacting the composition formed after step (ii) with a bioluminescent protein substrate and optionally a co-factor prior.

In alternative embodiments, there is provided a method of detecting a hydrolase in a sample, the method comprising (i) contacting a sample with a blocked non-protein acceptor domain having the structure B—R² to form a treated sample; (ii) contacting the treated sample with a compound of formula R¹-L or L-R¹ under conditions to cause attaching of R² to L; and (iii) detecting a change in BRET ratio, wherein the change in the BRET ratio corresponds to the presence of a hydrolase in the sample and the formation of a compound of formula R¹-L-R² or R²-L-R¹ and wherein R¹ is a bioluminescent protein; L is a linking element; R² is a non-protein acceptor domain; and B is a blocking group comprising a hydrolysable bond (see, for example, FIG. 10B). L, R² and B are all defined herein before. In some embodiments, the method further comprises contacting the composition formed after step (ii) with a bioluminescent protein substrate and optionally a co-factor prior to step (iii). For example, in some embodiments there is provided a method of detecting an esterase in a sample, the method comprising (i) contacting a sample with a blocked non-protein acceptor domain having the structure B—R² to form a treated sample; (ii) contacting the treated sample with a compound of formula R¹-L or L-R¹ under conditions to cause attaching of R² to L; and (iii) detecting a change in BRET ratio, wherein the change in the BRET ratio corresponds to the presence of a hydrolase in the sample and the formation of a compound of formula R¹-L-R² or R²-L-R¹ and wherein R¹ is a bioluminescent protein; L is a linking element; R² is a non-protein acceptor domain; and B is a blocking group and R² bound to B comprises a hydrolysable bond. R¹, L, R² and B are all defined herein before. An advantage of these embodiments is that the blocking group (and accordingly the hydrolysable bond) can easily be varied in order to yield BRET sensors responsive to a range of hydrolytic enzymes optionally with different colour outputs optimised for different applications. These embodiments may also be useful when practical applications require it (for example, hindrance of the sensor for a specific enzyme, instability of enzyme reactive fluorescent tag and the like). As would be understood by a person skilled in the art, contacting a sample with a blocked non-protein acceptor domain having the structure B—R² to form a treated sample occurs under conditions that are suitable for hydrolysis of the hydrolysable bond by the hydrolase.

As the skilled person would appreciate, the sensors of the present invention can also be used to quantify the amount of hydrolase present in a sample. For example, in some embodiments the methods further comprise determining the concentration and/or activity of the hydrolase in the sample.

As the skilled person would be aware, the sensors of the present invention can also be multiplexed. In this system, two or more different sensor molecules are provided which are cleaved by different hydrolases. For example, a sensor of the present invention can be multiplexed with a sensor that is cleaved by bovine plasmin (see, for example, WO 2013/155553) and/or a sensor that is cleaved by a Pseudomonas spp. protease. In some embodiments, each different sensor molecule may include a different donor and/or acceptor molecule such that they emit at different wavelengths to enable the detection and quantification of different target compounds. In some embodiments, each different sensor molecule may have the same donor and/or acceptor molecule. In some embodiments, a single fluidic detection chamber is used. In alternative embodiments, a multi-channel detection device may be used.

The methods of the present invention can be performed on any system suitable for detecting a change in BRET ratio. As the person skilled in the art will appreciate the methods of the present invention can be performed in a batch (for example batch format using a plate reader) or flow format. For example, the methods of the present invention can be performed in a microplate format using a microplate reader equipped with the appropriate filters. The methods of the present invention can also be performed on a microfluidic device, such as described in WO 2013/155553. An example of a BRET based assay performed on a microfluidics device (the CYBERTONGUE device) is provided in PCT/AU2018/050824.

As would be understood by a person skilled in the art, the sensors, compositions and kits of the present disclosure may also be used for measuring the activity of a hydrolase and/or determining the concentration of a hydrolase. The sensors, compositions and kits of the present disclosure may also be used for detecting, measuring and/or determining the concentration of activators or inhibitors of hydrolases.

The sensors and compositions described herein may be used for monitoring hydrolase activity, in the food, beverage, animal health and human health diagnostics fields, for process control in food, chemical, biochemical and biopharmaceutical manufacture and processing and for monitoring bioremediation. In one example, the sensors and compositions described herein can be used for the detection of nerve agents. In this example, the sensor may be a substrate for a cholinesterase. In another example, the sensors and compositions described herein can be used for early diagnosis of mastitis in dairy cattle through detection of alkaline phosphatase activity in milk. In this example, the sensor may be a substrate for an alkaline phosphatase. In another example, the sensors and compositions described herein can be used for assessing the effectiveness of milk pasteurisation through detection of phosphatase activity in milk samples. In this example, the sensor may be a substrate for an alkaline phosphatase. In another example, the sensors and compositions described herein can be used to measure lipase activity levels, such as lipase activity levels in blood for early diagnosis of pancreatic pathologies. In this example, the sensor may be a substrate for an esterase, for example a lipase.

Sample

As described above, the sensors of the present invention can be used to detect the presence or absence of a hydrolase in a sample. The sensors can also be used to quantify the hydrolase amount and/or activity in a sample. The “sample” can be any substance or composition that has the potential to contain a hydrolase. Typically, a sample is any substance known or suspected of comprising the hydrolase. In some embodiments, the sample may be air, liquid, a biological material, a veterinary sample, a clinical sample, soil, a plant sample or an extract thereof. In some embodiments, the sample is selected from the group consisting of air, liquid, biological material, and soil or an extract thereof. The sample can also be an instrument.

In some examples, the sample comprises a biological material. As used herein, “biological materials” is defined broadly and includes any material derived in whole or in part from an organism. Biological materials include, but are not limited to, bodily fluids, cells, soft tissues (such as connective and non-connective tissue) and hard tissues (such as bone and cartilage). In some embodiments, the bodily fluids are blood, serum, sputum, mucus, pus, peritoneal fluid, urine, tears, faeces, sweat or other bodily fluids. In some embodiments, such materials may have been harvested or collected from a living organism and then subjected to further processing and/or chemical treatment. In an embodiment, the sensor is not used to detect a hydrolase within a living cell. Biological materials includes plant materials, animal materials, bacterial materials, and the like or an extract thereof.

In some embodiments, the sample comprises a clinical sample. Clinical samples includes but is not limited to blood, serum, sputum, mucus, pus, tears, faeces, sweat, peritoneal fluid and other bodily fluids.

In some examples, the sample comprises a dairy product. As used herein, the term “dairy product” includes milk and products derived partially or in full from milk. The milk may be obtained from any mammal, for example cow, sheep, goat, horse, camel, buffalo, human and the like. Dairy products include, but are not limited to, raw milk, low fat milk, skim milk, pasteurized milk, UHT milk, lactose-modified UHT milk, fortified UHT milk, flavoured UHT milk, and combinations of these products as well as UHT infant formula, cheese, yoghurt, whey, buttermilk, cream, milk powder, powdered infant formula and butter and the like. In some examples, the sample is milk or diluted milk. The dairy product may also be an extract, such as a partially purified portion, of dairy product comprising, or suspected of comprising, the carbohydrate of interest.

In some embodiments, the sample is selected from the group consisting of soil or an extract thereof, samples (e.g. swab, rinse and the like) from medical equipment, samples from machinery (e.g. swab, rinse and the like), samples from food processing equipment (e.g. swab, rinse and the like), and the like. In some embodiments, food processing equipment includes, but is not limited to, transport tankers, holding tanks, processing machinery, lines, tubing, connectors, valves and the like. The sample may be derived (for example a swab, rinse or the like) from machinery. Machinery includes any machinery suspected or known to harbour the hydrolase of interest and/or bacteria expressing the hydrolase of interest, for example any machinery involved in the production, storage and processing of a dairy product. In some embodiments, machinery includes, but is not limited to, buffer and holding silos, welded joints, buffer tank outlets, conveyer belts, ultrafiltration membranes, valves, air separators, tanker trucks, tanker truck storage tanks, storage tanks, gaskets, connecting pipes and the like. The sample may also be derived from medical equipment, for example the sample may be swabs or rinses from medical equipment including, but not limited to, catheters, intravenous lines, ventilators, wound dressings, contact lenses, dialysis equipment, medical devices and the like.

The sample may be obtained directly from the environment or source, or may be extracted and/or at least partially purified by a suitable procedure before a method of the invention is performed.

In some embodiments, the sample is an aqueous liquid. For example, the sample includes but is not limited to, milk, fruit juices, other beverages and bodily fluids including blood and serum.

In some embodiments, the sample may be a suspension or extract obtained by washing, soaking, grinding or macerating a solid agricultural, food or other substance in an aqueous solution and using the liquid phase as sample. The liquid phase sample may be clarified by any suitable technique, for example settling, filtration or centrifugation.

In some embodiments, the sample may be obtained by bubbling an air or other gas phase sample through an aqueous phase, or spraying the aqueous phase through an air or other gas phase or otherwise allowing the transfer of molecules from an air or other gas phase sample to an aqueous phase. The resulting aqueous phase would then be used as a sample for analysis.

### EXAMPLES

### Example 1—Construction of Sensor Molecules

A sensor molecule for measuring esterase activity was designed. The sensor comprises RLuc8 covalently attached via an N-terminal peptide linking element to the synthetic fluorescent probe fluorescein with acetate as the blocking group. The acetate blocking groups stabilise fluorescein in a non-fluorescent state until the ester bond is cleaved by an esterase. Consequently, BRET from the donors to the small molecule fluorophore is only observed following removal of the acetate groups by an esterase and activation of the fluorescein acceptor.

Materials and Methods

Production of Wt-RLuc8 and RLuc8Cys Variants 1, 2 and 3

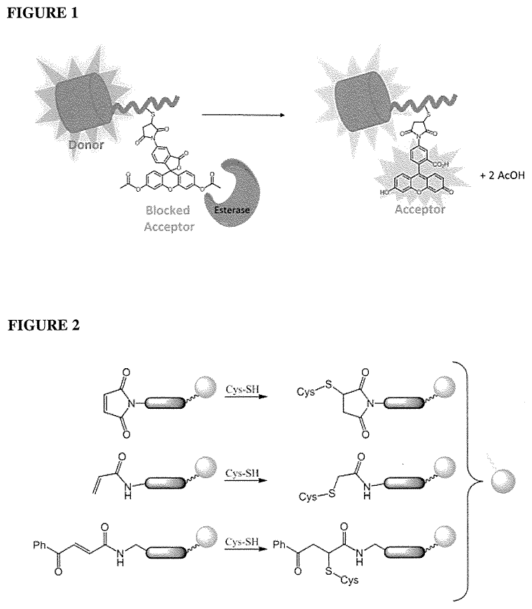

In the exemplified sensors, RLuc8 is connected through an N-terminal peptide linking element to a synthetic fluorophore (FIG. 1). In order to allow specific tagging of the linking element, a single Cys residue was introduced within the peptide linker. Although two Cys residues are endogenous to RLuc8, a Cys residue was introduced into the linking element to provide increased availability for reaction with the fluorescent acceptor domain and/or hydrolase.

pRSET RLuc8 PCR encodes RLuc8 (SEQ ID NO: 1) preceded by an N-terminal linking element having the sequence shown in SEQ ID NO: 7. Single cysteine residues were introduced at various positions in N-terminal peptide linking element by PCR using pRSET RLuc8 as the template and with the appropriate primers (Table 4). Mutagenesis of pRSET-RLuc8 was carried out according to a published procedure (Zheng et al., 2004). Plasmids encoding RLuc8Cys1, 2 and 3 (SEQ ID NO: 12-14) were identified and confirmed by DNA sequencing.

TABLE 4
Oligonucleotides used in the preparation
of pRSET RLuc8 Cys mutants.

| | Orien- | Oligo 5′-3′ | Location |
|---|---|---|---|
| Mutant | tation* | sequence | of Cys** |
| RLuc8Cys1 | F | ATGGGGATCCGAATG | 1 aa |
| | | CATGGCTTCCAAGG | |
| | | (SEQ ID NO: 15) | |
| | R | CCTTGGAAGCCATGC | |
| | | ATTCGGATCCCCAT | |
| | | (SEQ ID NO: 15) | |
| RLuc8Cys2 | F | GGATCTGTACGACTG | 11 aa |
| | | CGACGATAAGGATCG | |
| | | (SEQ ID NO: 17) | |
| | R | CGATCCTTATCGTCG | |
| | | CAGTCGTACAGATCC | |
| | | (SEQ ID NO: 18) | |
| RLue8Cys3 | F | CTAGCATGACTGGTT | 21 aa |
| | | GCCAGCAAATGGGTC | |
| | | (SEQ ID NO: 19) | |
| | | GACCCATTTGCTGGC | |
| | R | AACCAGTCATGCTAG | |
| | | (SEQ ID NO: 20) | |

*F is forward primer; R is reverse primer.
**Number of amino acids between the N-terminal residue of RLuc8 and the introduced cysteine.

Wild-type (wt) RLuc8 and the cysteine variants, RLuc8Cys1, 2 and 3, were expressed in E. coli BL21(DE3) (New England BioLabs). An overnight culture was grown from a single colony in LB (10 g tryptone, 5 g yeast extract, 5 g NaCl (pH 7.4) per L) containing 100 μg/mL ampicillin and 2% glucose at 37° C., 200 rpm. The overnight culture was used to inoculate 250 mL LB (100 μg/mL ampicillin) to an OD₆₀₀ of 0.05 and the culture was incubated at 37° C., 200 rpm for 4.5 hours. Protein expression was induced by reducing the temperature to 22° C. and incubating overnight at 200 rpm. Cells were harvested by centrifugation (5000×g, 10 min, 4° C.) 24 hours after inoculation. The supernatant was removed and the cell pellet washed with PBS before being resuspended in 50 mM NaPi, 0.3 M NaCl, pH 7.0. Cells were disrupted using a homogenizer (Microfluidics M-110P) at P=20 000 psi and the soluble fraction was isolated by centrifugation (15 000×g, 15 min, 4° C.). His₆-tagged proteins were isolated using cobalt affinity chromatography (TALON® Superflow Metal Affinity Resin (Takara Clontech, Australia)) according to the manufacturer's instructions. Following elution with 150 mM imidazole solution, the protein was dialyzed against MES buffer (50 mM MES, 300 mM NaCl, 0.1 mM EDTA, pH 6.0) using a dialysis unit (GE Healthcare, Vivaspin 6, 10 kDa MWCO). 500 μL aliquots of the purified protein were snap-frozen in liquid nitrogen and stored at −80° C. Protein concentrations were determined by absorbance at 280 nm.

Labelling of RLuc8 Cysteine Variants with Fluorescein Analogues

wt-RLuc8 or RLuc8Cys 1, 2 and 3 variant (5 μM) in 50 mM MES, pH 5.0 was incubated with 4× molar excess (20 μM) of fluorescein analogue (from 1 mM stock in DMSO) and the mixture was shaken gently at 4° C. for the indicated time (6 to 60 minutes). At the end of incubation time, the reaction mixture was buffer exchanged by centrifugation (10 kDa MWCO, 13000×g, 13 min, 4° C.) or desalting columns (HiTrap™ desalting, GE Healthcare) to remove the excess labelling agent. Bioluminescence spectra as described below were recorded shortly following labelling.

BRET Assays

BRET assays were carried out in 96-well plates (Perkin-Elmer, Australia) with a final volume of 100 μL. 1 μM of purified protein was used for all BRET assays, in a final volume of 100 μL, where the protein was diluted in PBS or MES as required.

For BRET measurement, 5 μL of coelenterazine 400a in EtOH was added to the protein sensor solution (final [coel 400a]=16.7 μM) and spectral scans were recorded immediately. Spectral scans were recorded with a Spectramax M2 plate-reading spectrofluorimeter (Molecular Devices). Bioluminescence scans were recorded using luminescence scan mode, between 380-600 nm, at 20 nm intervals.

Data Analysis

BRET² ratios were calculated as the ratio of the maximum acceptor emission intensity (520 nm) to maximum donor emission intensity (420 nm).

Results

The esterase sensor was prepared by labelling the N-terminal peptide linker of RLuc8Cys2 with the hydrolysable fluorescein diacetate-5-maleimide. Labelling conditions were optimised to maximise labelling efficiency of the N-terminal peptide linker of RLuc8, while minimising chemical hydrolysis of the acetate groups of the fluorescein derivative Minimising chemical hydrolysis of the tag prior to enzymatic assay reduces the background fluorescence of the sensor and increases the sensitivity of enzyme detection.

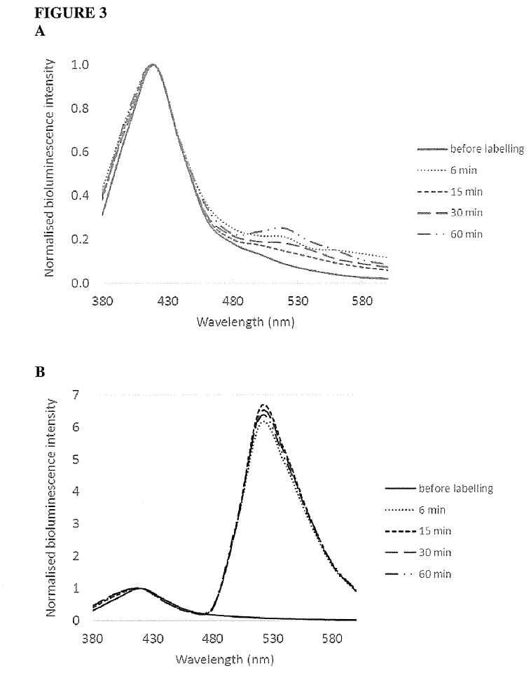

To determine optimal labelling conditions, labelling of both wt-RLuc8 and RLuc8Cys variants was carried out using fluorescein-5-maleimide with varying incubation times. The excess labelling agent was removed by filtration and bioluminescence spectra were recorded. The BRET ratios measured were used as an indication of the labelling efficiency over time for wt-RLuc8 (FIG. 3A) and RLuc8Cys1 (FIG. 3B). As presented in FIG. 3, labelling of RLuc8Cys1 with fluorescein yielded BRET ratios of approximately 6.5 for all incubation times tested (FIG. 3B), indicating that the labelling reached completion within 6 minutes. For the present sensor, the preferred labelling time was 6 min.

Although very poor BRET ratios were measured for wt-RLuc8 labelling (FIG. 3A), a slight increase in BRET ratio was observed for labelling times between 6 and 60 minutes. This indicates that, while quantitative labelling of the side chain Cys is achieved within minutes, prolonging the incubation time has the potential to increase labelling of the endogenous Cys residues.

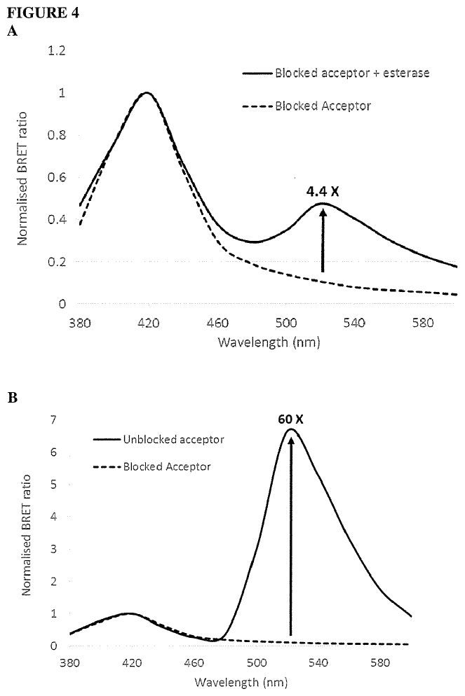

As presented in FIG. 4A, labelling of RLuc8Cys2 with fluorescein diacetate 5-maleimide gave a very low BRET ratio of 0.11 (solid line). The low BRET level observed with the ‘blocked’ small-molecule acceptor indicates that the optimised labelling and purification conditions are suitable to yield an esterase BRET sensor with minimal background fluorescence.

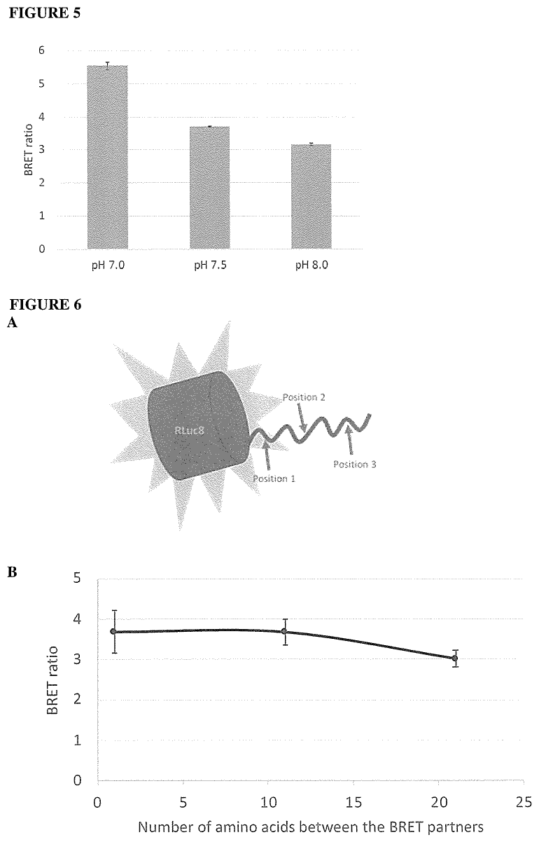

The pH dependence of the BRET ratio is presented in FIG. 5. The BRET ratio is highest at pH 7.0.

### Example 2—Linker Length

In order to assess the effect of the length of the linking element on BRET for the exemplified sensor molecule, cysteine residues were introduced into the N-terminal linking element 1 amino acids, 11 amino acids and 21 amino acids from the N-terminus of RLuc8 (Table 4; FIG. 6A). The RLuc8Cys variants (namely, RLuc8Cys1, 2 and 3) were labelled with fluorescein-5-maleimide according to the optimised protocol described in example 1 and the BRET ratios were measured as described in to example 2 (FIG. 6B). As shown in FIG. 6B, the BRET ratio decreases as the number of amino acids between the RLuc8 and fluorescein increases. Of the three sensors tested, the RLuc8Cys2 sensor was chosen for further investigation as it provided near maximum BRET ratio but with a longer linking element which is thought to improve the accessibility of the hydrolysable bond.

### Example 3—Measurement of Esterase Activity Using the RLuc8Cys2 Sensor

In order to determine whether RLuc8Cys2 can be used to detect and measure the activity of an esterase, the sensor was reacted with porcine liver esterase (PLE; 8 U/mL) to hydrolyse the acetate groups and free the fluorescent acceptor. Briefly, 1 μM RLuc8Cys2, diluted in a final volume of 100 μL MES pH 5.0 was incubated with PLE (0.8 U) for 10 minutes at 37° C. At the end of the incubation time, 5 μL of coelenterazine 400a in EtOH was added to the protein sensor solution (final [coel 400a]=16.7 μM) and spectral scans were recorded immediately as described in example 2.

As presented in FIG. 4A, treatment of the esterase sensor with PLE yielded a partially unblocked acceptor, increasing the BRET ratio of 0.11 to 0.47, a 4.4 fold increase (FIG. 4, dotted line). It is noteworthy that although a 4.4 fold BRET increase was observed under the hydrolysis conditions used, a maximal BRET ratio of 6.6 (FIG. 4B, dotted line) can potentially be obtained, representing a potential dynamic range of up to 60 fold.

### Example 4—Cloning, Expression and Purification of Sensor Molecules

Materials and Methods

Cloning of RLuc8Cys Variants 4 and 5 and MBP(K239C)RLuc8

A single Cys residue was introduced within the peptide linker at position 2 and 11 (i e immediately following the His₆ tag) by PCR using pRSET RLuc8 as the template and with the appropriate primers (Table 5). Mutagenesis of pRSET-RLuc8 was carried out according to a published procedure (Zheng et al., 2004). Plasmids encoding RLuc8Cys4 and 5 (SEQ ID NO: 35 and 36) were identified and confirmed by DNA sequencing.

In order to investigate the effect of a larger distance between the donor and acceptor domains, maltose binding protein was cloned into pRSET RLuc8 (between the sequence encoding the N-terminal histidine tag and RLuc8) forming pRSET MBP RLuc8. A lysine residue (K289) predicted to be on the surface of MBP was mutated to a cysteine to allow labelling with a fluorescent acceptor domain Mutagenesis was carried out by PCR using pRSET MBP RLuc8 as the template and with the appropriate primers (Table 5) (Zheng et al., 2004). Plasmids encoding MBP(K289C)RLuc8 (SEQ ID NO: 34) were identified and confirmed by DNA sequencing.

All constructs were expressed with an N-terminal hexa-histidine tag.

Expression and Purification of the RLuc8 Sensors

Wild-type (wt) RLuc8, the cysteine variants, RLuc8Cys1, 2, 3, 4 and 5 and MBP(K239C)RLuc8 were expressed in E. coli BL21(DE3) (New England BioLabs). An overnight culture was grown from a single colony in LB (10 g tryptone, 5 g yeast extract, 5 g NaCl (pH 7.0) per L) containing 100 μg/mL ampicillin and 2% glucose at 37° C., 200 rpm. The overnight culture was used to inoculate 250 mL LB (100 μg/mL ampicillin) to an OD₆₀₀ of 0.05 and the culture was incubated at 37° C., 200 rpm for 4.5 hours. Protein expression was induced by reducing the temperature to 22° C. and incubating overnight at 200 rpm. Cells were harvested by centrifugation (4000×g, 10 min, 4° C.). The supernatant was removed and the cell pellet washed with PBS before being resuspended in 50 mM NaPi, 0.1 M NaCl, pH 7.0. Cells were disrupted using a homogenizer (Microfluidics M-110P) at P=20 000 psi and the soluble fraction was isolated by centrifugation (15 000×g, 15 min, 4° C.). His₆-tagged proteins were isolated using cobalt affinity chromatography (TALON® Superflow Metal Affinity Resin (Takara Clontech, Australia)) according to the manufacturer's instructions. Following elution with 150 mM imidazole, 50 mM NaPi, 0.1 M NaCl (pH 7.4), the protein was dialyzed against MES buffer (50 mM MES, 50 mM NaCl, pH 7.5) using a dialysis unit (Novagen, D-Tube™ Dialyzer Mega, MWCO 6-8 kDa). The purified protein was snap-frozen in liquid nitrogen and stored at −80° C. Protein concentrations were determined using Bradford methodology (Sigma Aldrich protocol).

TABLE 5
Oligonucleotides used in the preparation
of pRSET RLuc8 Cys mutants.

| | | | Location |
|---|---|---|---|
| | Orien- | Oligo 5’-3’ | of |
| Mutant | tation* | sequence | Cys** |
| RLuc8Cys4 | F | TCATCATCATCATCATT | 28 aa |
| | | GCATGGCTAGCATGAC | |
| | | (SEQ ID NO: 38) | |
| | R | GTCATGCTAGCCATGCA | |
| | | ATGATCATGATGATGA | |
| | | (SEQ ID NO: 39) | |
| RLuc8Cys5 | F | AAGGAGATATACATATG | 37 aa |
| | | TGCGGTTCTCATCATCAT | |
| | | (SEQ ID NO: 40) | |
| | R | ATGATGATGAGAACCGCA | |
| | | CATATGTATATCTCCTT | |
| | | (SEQ ID NO: 41) | |
| MBP(K239C) | F | TGGTCCAACATCGACTCC | −7.1 nm |
| | | ACCAAAGTCAATTATGG | from the |
| | | (SEQ ID NO: 42) | centre |
| | | | of |
| | R | AACATCGACACCAGCTGC | RLuc8*** |
| | | GTGAATTATGGTGTAAC | |
| | | (SEQ ID NO: 43) | |

*F is forward primer; R is reverse primer.
**Number of amino acids between the N-terminal residue of RLuc8 and the introduced cysteine.
***The distance between the cysteine residue at position 239 and the centre of RLucS was estimated using CLCsequence view 8 available from Qiagen and the crystal structure of MBP (PDB ID: 1ANF; Quiocho ct al.. 1997).

Bioconjugation of RLuc8 Variants

RLuc8 or RLuc8 variant (10 μM) in 8:2 MES:HEPES, pH 5.5 (50 mM MES, 50 mM NaCl, pH 3.6; 50 mM HEPES, 50 mM NaCl, pH 7.5) was incubated with 10 eq (100 μM) of fluorescein-5-maleimide (FM; Sapphire Bioscience), sulforhodamine B,C₂-maleimide (RM; Serateh Biotech, USA) or fluorescein-diacetate-6-maleimide (FD; Sapphire Bioscience) (from 10-20 mM stock in DMSO) at 25° C. for 5 to 60 minutes. At the end of incubation time, the RLuc8 bioconjugate was purified on HiTrap™ Desalting column (GE Healthcare) to remove the excess labelling agent. MES pH 5.0 (50 mM MES, 50 mM NaCl, pH 5.0) was used as the elution buffer. RLuc8 bioconjugate were snap frozen in liquid nitrogen and stored at −80° C.

RLuc8 Bioconjugate Quantification

5 μL of BSA standards (0, 0.1, 0.3, 0.6, 1.0, 1.4 mg/mL) in buffer (50 mM MES, 50 mM NaCl, pH 5.0) or RLuc8 bioconjugate was dispensed in separate wells of a clear 96-well plate, in triplicate. 250 μL of room temperature Bradford reagent (Sigma Aldrich) was added to each well and the plate gently mixed for 30 seconds. The reaction mix was incubated at room temperature for 10 minutes and the absorbance at 595 nm (A₅₉₅) was measured. A standard curve was constructed by plotting the A₅₉₅ of the samples was plotted against the BSA standard concentrations. The RLuc8 bioconjugate concentrations were determined by comparing the net A₅₉₅ values against the standard curve.

SDS-PAGE of RLuc8 Bioconjugates

RLuc8 bioconjugate (5 μg) and NuPage LDS sample buffer 4× (ThermoFisher) were mixed and the samples were incubated at 98° C. for 5 minutes. Protein samples were loaded on NuPage bis-tris gel (ThermoFisher) and ran at 200 V for 40 min Fluorescent gel was recorded in gelDoc (5 msec exposition) and stained in Coomassie BullDog stain.

BRET Assay

BRET assays were carried out in 96-well plates (Perkin-Elmer, Australia) with a final volume of 100 μL. 1 μM of purified protein was used for all BRET assays, in a final volume of 100 μL, where the protein was diluted in 8:2 HEPES:MES, pH 7.5 (50 mM HEPES, 50 mM NaCl, pH 7.8; 50 mM MES, 50 mM NaCl, pH 5.0).

For BRET measurement, 1 μL of coelenterazine 400a in EtOH was added to the reaction mix (final [coel400a]=17 μM), shaken for 1 msec and spectral scans recorded immediately. Spectral scans were recorded with a Spectramax M3 plate-reading spectrofluorimeter (Molecular Devices). For fluorescein based sensor, bioluminescence scans were recorded using luminescence scan mode, between 360-600 nm, at 20 nm intervals. For rhodamine based sensor, bioluminescence scans were recorded using luminescence scan mode, between 360-700 nm, at 20 nm intervals.

Data Analysis

BRET² ratios were calculated as the ratio of the maximum acceptor emission intensity (520 nm (fluorescein) or 600 nm (rhodamine)) to maximum donor emission intensity (420 nm).

Results

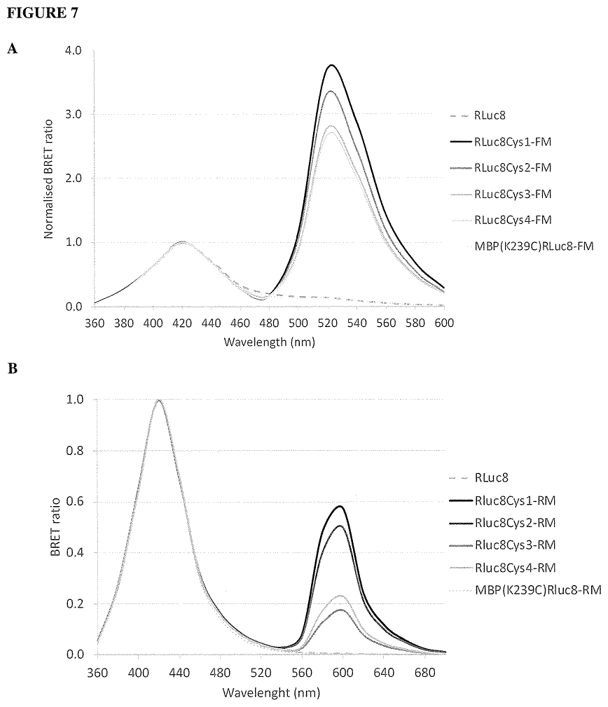

In order to further assess the effect of the length of the linking element on BRET for the exemplified sensor molecules, cysteine residues were introduced into the N-terminal linking element at position 2 (RLuc8Cys5; SEQ ID NO: 33) and position 11 (RLuc8Cys4; SEQ ID NO: 32). A MBP(K239C)RLuc8 fusion was also generated to assess the impact of a larger gap between the donor and acceptor domains (Table 5). The RLuc8Cys variants were labelled with fluorescein-5-maleimide (FM) or sulforhodamine B,C₂-maleimide (RM) and BRET spectra were measured for the FM (FIG. 7A) and RM variants (FIG. 7B). The BRET ratio for the FM and RM variants was also calculated (FIG. 7C). As shown in FIG. 7, the BRET ratio decreases as the number of amino acids between the donor and acceptor increases. As is also shown in FIG. 7, the BRET ratio is greater for the FM variants and these variants were chosen for further investigation. However, the BRET ratio of the RM variants indicates that rhodamine would be suitable for use in the sensors of the present application.

### Example 5—Measurement of Esterase Activity

Materials and Methods

In a white 96-well plate, 1 μM of the RLuc8Cys(variant)-fluorescein diacetate sensor (RLuc8Cys-FD)) was incubated with 2.9 U of Porcine Liver Esterase (PLE) (Sigma-Aldrich #E3019) for 10, 20, 40 or 60 minutes at various temperatures (Table 6). The final reaction mix contained 20% 40 mM MES, 50 mM NaCl, pH 5.0 and 80% of the buffer described in Table 6.

At the end of the incubation time, 1 μL of coelenterazine 400a in EtOH was added (final [coel400a]=17 μM) and spectral scans were recorded immediately as described in Example 4. To assess chemical hydrolysis of the sensor, the same assay was performed in the absence of PLE. Data were corrected for chemical hydrolysis of the sensor. Experiments at pH 7.0, pH 7.5 and pH 8.0 were carried out for 20 minutes only. Experiments at pH 6.0 and pH 6.5 were monitored over 20, 40 and 60 minutes.

TABLE 6
Buffer used for esterase assay using RLuc8Cys-FD

| Temperature | pH | Buffer |
|---|---|---|
| 30° C. | 6.0 | 50 mM MES, 50 mM NaCl (pH 6.3) |
| | 6.5 | 50 mM MES, 50 mM NaCl (pH 6.8) |
| | 7.0 | 50 mM HEPES, 50 mM NaCl (pH 7.3) |
| | 7.5 | 50 mM HEPES, 50 mM NaCl (pH 7.8) |
| | 8.0 | 50 mM HEPES, 50 mM NaCl (pH 8.3) |
| 25° C. | 6.5 | 50 mM MES, 50 mM NaCl (pH 6.8) |
| | 7.0 | 50 mM HEPES, 50 mM NaCl (pH 7.3) |
| 20° C. | 7.0 | 50 mM HEPES, 50 mM NaCl (pH 7.3) |

Results

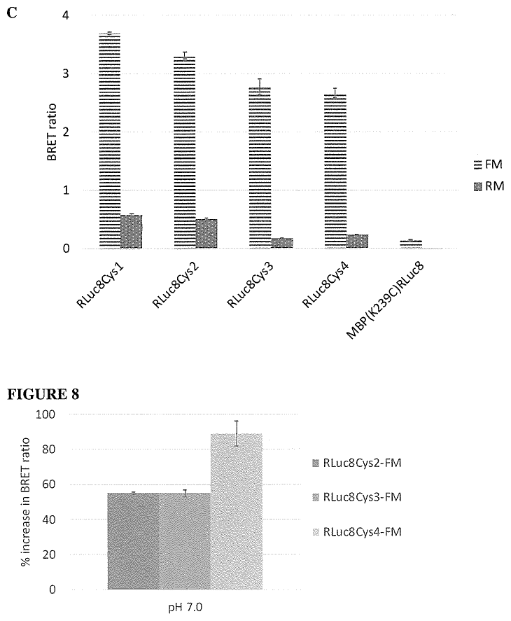

Initial experiments characterised the ability of the RLuc8Cys2-fluorescein-diacetate sensor, RLuc8Cys3-fluorescein-diacetate sensor and RLuc8Cys4-fluorescein-diacetate sensor to detect and measure the activity of the esterase, PLE, at 20° C. and pH 7.0. As shown in FIG. 8, the percentage increase in BRET ratio was greatest for RLuc8Cys4-fluorescein-diacetate sensor. Without wishing to be bound by theory, it was thought that the ester linkage was more accessible in the RLuc8Cys4-fluorescein-diacetate sensor. Accordingly, the RLuc8Cys4-fluorescein-diacetate sensor was chosen for further investigation.

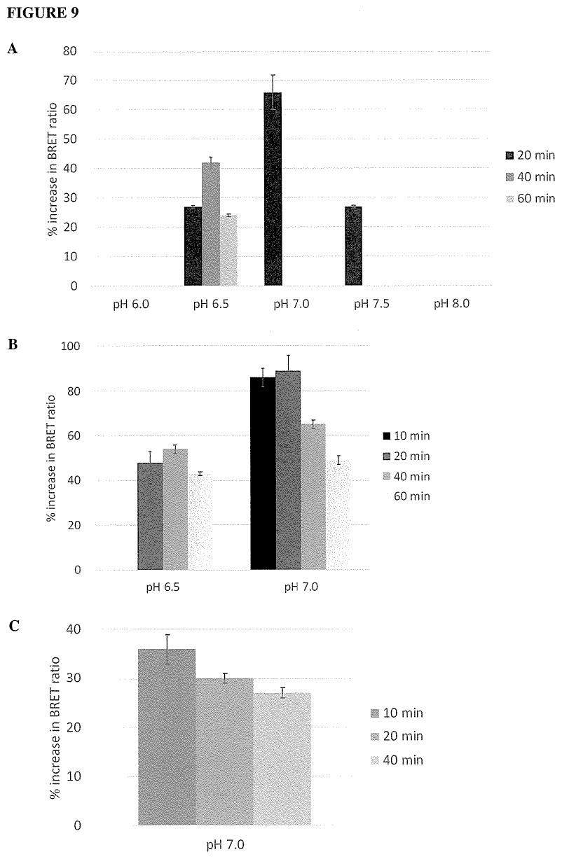

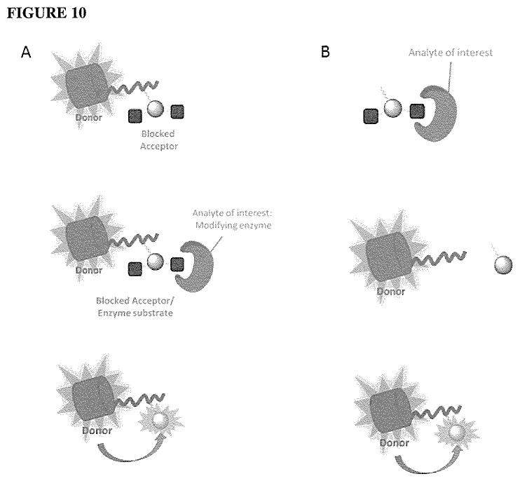

To further characterise the ability of the RLuc8Cys4-fluorescein-diacetate sensor to detect and measure the activity of an esterase, the sensor was reacted with PLE at various pH and temperature. As presented in FIG. 9, treatment of the RLuc8Cys4-fluorescein-diacetate sensor with PLE yielded a partially unblocked acceptor, increasing the BRET ratio at pH 6.5, 7.0 and 7.5. At pH 6.0, the activity of PLE was undetectable consistent with the known pH dependence of PLE. At pH 8.0, the activity of PLE was undetectable as the rate of chemical hydrolysis of the sensor was higher than the rate of esterase activity. Without wishing to be bound by theory, it is thought that the lack of linearity of the % increase in BRET ratio over time is also a result of chemical hydrolysis of the sensor. It is also possible that the esterase enzyme is losing activity over time relative to the rate of chemical hydrolysis (background hydrolysis).

### Example 6—Measurement of Phosphatase Activity for Assessing the Effectiveness of Milk Pasteurisation

Phosphatases (EC 3.1.3.x) are a subclass of hydrolases that catalyse the hydrolysis of phosphomonoesters. Phosphatase enzymes are almost ubiquitous in nature being involved in nucleic acid transformations, postranslational modifications of proteins and many reactions of bioenergetics and secondary metabolism. Phosphatase activity, or the effect of inhibitors of phosphatase activity, may be conveniently measured using the sensors defined in the present disclosure. For example, alkaline phosphatase (EC 3.1.3.1) is a widely distributed phosphatase and measurement of its activity is frequently used as a proxy for a range of medical and other diagnostic purposes. For example, measurement of residual alkaline phosphatase activity can be used to assess the effectiveness of pasteurisation of raw milk (Kay, 1935; Hoy and Neave, 1937; Rankin et al., 2010) because the temperature-time profile required to inactivate the alkaline phosphatase activity naturally present in raw milk is slightly more stringent than is required to inactivate the main pathogens potentially present in milk Therefore a phosphate-blocked sensor of the type described herein (see, for example, Table 1) can be applied to determining the effectiveness of pasteurisation of milk, by measuring the residual level of alkaline phosphatase after treatment, or before and after treatment.

Materials and Methods

BRET assays would be carried out in 96-well plates with a final volume of 100 μL. Spectral scans would be recorded with a SpectraMax M3 plate-reading spectrofluorimeter (Molecular Devices) in luminescence mode (20 nm increments) in white 96-well plates (Opti-Plate™-96, PerkinElmer).

1 μM of a sensor molecule defined herein, such as a sensor molecule wherein R¹ comprises RLuc8, L comprises a 28 amino acid polypeptide comprising a cysteine residue, R² bound to B is fluorescein phosphate or fluorescein diphosphate (as shown in Table 1) and R² bound to B is attached to the cysteine residue on the 28 amino acid polypeptide via a maleimide linking group. The sensor would be diluted to the desired concentration using a suitable buffer such as 100 mM TrisHCl, 68 mM NaCl, pH 8.0 and 45 μL of this preparation would be mixed with 50 μL of milk. Any suitable milk may be used in the assay, for example raw cow's milk as a control, or pasteurized milk, which could be otherwise unmodified or have modified levels of fat and/or protein, and/or lactose or indeed be subject to additional heat treatment or additions (such as flavours or colours). The mixture of milk and sensor would be incubated for a time period of between 1 and 120 minutes, typically between 5-10 minutes at 20-30° C. At the end of the incubation time, 5 μL of coelenterazine 400a in EtOH would be added (to a final coelenterazine 400a concentration of 17 μM) making up the reaction to a final volume of 100 μL and the spectral scans would be recorded immediately. The BRET ratio would be calculated as the ratio of the peak fluorescent acceptor emission intensity to the peak donor emission intensity, which for RLuc would typically be at 420 nm. Alternatively, the intensity of the donor and acceptor emissions can be measured in an instrument with bandpass or other spectral filters such as a Clariostar plate reader (BMG Labtech) and the BRET ratios calculated as the ratio of RLuc emission intensity to fluorescent acceptor emission intensity. This assay can also be performed on a microfluidic device, such as described in WO 2013/155553 and PCT/AU2018/050824.

Data Analysis and Interpretation of Results

The effectiveness of pasteurisation would be assessed by comparison with the change in BRET ratio typically observed using known amounts of alkaline phosphatase and/or samples of unpasteurised raw milk (which has high levels of alkaline phosphatase) and samples of authentically pasteurised or even UHT milk (which have very low or undetectable levels of alkaline phosphatase). Successful pasteurisation would have low or undetectable levels of alkaline phosphatase. Therefore, when the milk has been successfully pasteurised high levels of RLuc donor emission intensity and low levels of fluorescent acceptor moiety emission intensity, corresponding to a low BRET ratio, would be observed. In the case of unsuccessful pasteurisation or contamination of pasteurised milk with unpasteurised milk, lower levels of donor peak emission intensity and higher levels of acceptor peak emission intensity, corresponding to elevated or high BRET ratios, would be observed. Even moderate elevation of the BRET ratio above the negative control samples would be considered a cause for concern, indicating incomplete pasteurisation or contamination with unpasteurised milk.

### Example 7—Measurement of Phosphatase Activity for Diagnosing Pre-Clinical or Clinical Mastitis

Pre-clinical and clinical mastitis in cows is associated with an elevation of alkaline phosphatase (EC3.1.3.1) in the milk and that this may be localised to milk from the quarter or quarters with inflammation (Bogin and Ziv, 1973). Research has indicated that measuring alkaline phosphatase in the milk of Holstein cows has sufficient sensitivity and specificity to be used to diagnose subclinical mastitis in individual cows (Babaei et al., 2007). Therefore a phosphate-blocked sensor of the type described herein (see, for example, Table 1) can be used to determine the likelihood of an individual cow or a specific quarter from a cow experiencing pre-clinical mastitis or mastitis.

Materials and Methods

BRET assays would be carried out in 96-well plates with a final volume of 100 μL. Spectral scans would be recorded with a SpectraMax M3 plate-reading spectrofluorimeter (Molecular Devices) in luminescence mode (20 nm increments) in white 96-well plates (Opti-Plate™-96, PerkinElmer).

1 μM of a sensor molecule defined herein, such as a sensor molecule wherein R¹ comprises RLuc8, L comprises a 28 amino acid polypeptide comprising a cysteine residue, and R² bound to B is fluorescein phosphate or fluorescein diphosphate (as shown in Table 1) and R² bound to B is attached to the cysteine residue on the 28 amino acid polypeptide via a maleimide linking group. The sensor would be diluted to the desired concentration using a suitable buffer such as 100 mM TrisHCl, 68 mM NaCl, pH 8.0. 45 μL of the sensor would be mixed with 50 μL of unmodified raw cow's milk Samples may be collected separately from each quarter of the udder, or alternatively samples may be combined from two or more quarters. The milk with the sensor would be incubated for between 1 and 120 minutes, typically between 5-10 minutes at 20-30° C. At the end of the incubation time, 5 μL of coelenterazine 400a in ethanol would be added (to a final coelenterazine 400a concentration of 17 μM) and spectral scans recorded immediately. The BRET ratio would be calculated as the ratio of the peak fluorescent acceptor emission intensity to the peak donor emission intensity, which for RLuc would typically be at 420 nm. Alternatively, the intensity of the donor and acceptor emissions could be measured in an instrument with bandpass or other spectral filters such as a Clariostar plate reader (BMG Labtech) and the BRET ratios calculated as the ratio of RLuc emission intensity to fluorescent acceptor emission intensity. This assay can also be performed on a microfluidic device, such as described in WO 2013/155553 and PCT/AU2018/050824.

Data Analysis and Interpretation of Results

The likelihood of mastitis or sub-clinical mastitis would be assessed by comparing the BRET ratio obtained when the assay is performed using the test sample (for example from a cow suspected of having mastitis) to the BRET ratio obtained when the assay is performed using a raw milk sample from healthy animals of the same herd or a previous milk collection from the same animal or, ideally, by comparison with the previous records of alkaline phosphatase activity measured in each quarter of each cow. This approach is feasible when using modern automated milking systems that routinely collect and analyse milk from individual quarters.

Elevated levels of alkaline phosphatase would result in lower levels of donor peak emission intensity and higher levels of acceptor peak emission intensity (corresponding to elevated or high BRET ratios as defined herein). A statistical threshold, such as an elevation in alkaline phosphatase activity of greater than or equal 10 to 1-3 standard deviations above the mean levels observed previously from that cow, or that quarter, could be used to determine when elevation of the BRET ratio would be considered a cause for concern and/or trigger a follow up.

### Example 8—Measurement of Lipase Activity

Lipases (EC 3.1.1.x) are a sub-class of esterases that hydrolyse esters formed between alcohols and medium to long chain fatty acids. Lipases are ubiquitous in nature and have found many important industrial and other uses. It is therefore important to measure lipase activity in a range of circumstances, including clinical diagnosis and as part of quality control in industrial processing and during formulation of commercial products containing lipases (Stoytcheva et al., 2012). Measurement of lipase activity can be achieved using the sensor defined herein where B is, for example, a medium to long chain fatty acid or an acyl or diacyl glycerol linked to the fluorophore via an acylester bond.

Materials and Methods

BRET assays would be carried out in 96-well plates with a final volume of 100 μL. Spectral scans would be recorded with a SpectraMax M3 plate-reading spectrofluorimeter (Molecular Devices) in luminescence mode (20 nm increments) in white 96-well plates (Opti-Plate™-96, PerkinElmer).

A sensor molecule as defined herein, such as a sensor molecule wherein R¹ comprises RLuc8, L comprises a 28 amino acid polypeptide comprising a cysteine residue, and R² bound to B is fluorescein laurate or fluorescein dilaurate and R² bound to B is attached to the cysteine residue on the 28 amino acid polypeptide via a maleimide linking group, would be diluted to a final concentration of between 2-5 μM using a suitable buffer (for example, 50-100 mM NaCl, 40-100 mM Tris-HCl, pH 8.0, 0.0125-0.05% (v/v) Zwittergent or Triton X-100 or an equivalent micelle forming detergent and 2-4% (w/v) fatty acid free bovine serum albumen (based on Basu et al., 2011). 45 μL of this preparation would be mixed with 50 μL of the lipase containing sample, and would be incubated for a time of between 1 and 120 minutes, typically between 5-10 minutes, at 20-30° C. At the end of the incubation time, 5 μL of coelenterazine 400a in EtOH would be added (to a final coelenterazine 400a concentration of 17 μM) and spectral scans would be recorded immediately. The BRET ratio would be calculated as the ratio of the peak fluorescent acceptor emission intensity to the peak donor emission intensity, which for RLuc would typically be at 420 nm. Alternatively, the intensity of the donor and acceptor emissions could be measured in an instrument with bandpass or other spectral filters such as a Clariostar plate reader (BMG Labtech) and the BRET ratios calculated as the ratio of RLuc emission intensity to fluorescent acceptor emission intensity. Alternatively, the intensity of the donor and acceptor emissions could be measured in an instrument with bandpass or other spectral filters such as a Clariostar plate reader (BMG Labtech) and the BRET ratios calculated as the ratio of RLuc emission intensity to fluorescent acceptor emission intensity. This assay can also be performed on a microfluidic device, such as described in WO 2013/155553 and PCT/AU2018/050824.

Prior to performing the assays, the lipase containing sample could be pre-incubated with specific lipase inhibitors, for example selected from those mentioned in Iglesias et al., 2016, as this would allow the specificity of the lipase assay to be tuned to just the lipase or lipases of interest.

The assay would be performed with potential lipase containing samples including, but not limited to, clinical samples or other types of biological samples or an industrial sample containing lipase(s) of interest.

Data Analysis and Interpretation of Results

The relative activities of particular lipases in the presence or absence of specific lipase inhibitors can be assessed by comparing the extent of sensor modification and therefore change in BRET ratio with the changes in BRET ratio brought about by known amounts of standard lipases under the same conditions. Under comparable conditions, higher levels of lipase activity would result in lower levels of donor peak emission intensity and higher levels of acceptor peak emission intensity (corresponding to elevated or high BRET ratios as defined herein).

### Example 9—Calculation of Esterase, Phosphatase or Lipase Activity

Esterase, Phosphatase or Lipase activity can be calculated from a change in BRET ratio as measured with a sensor defined herein. Enzyme activity can conveniently be expressed in relative terms, in this case the change in the BRET ratio over a specified time. Comparing the rates of change in BRET ratio (e.g. the numerical changes in BRET ratio over a 1 minute period) under standard assay conditions between samples and/or between samples and standards, and/or samples and positive and negative controls would be suitable for most practical applications of the esterase, phosphatase and lipase or other hydrolase sensors defined herein.

If it was desired to estimate results in absolute terms (i.e. micromoles of substrate converted per minute) the BRET based sensors defined herein can be calibrated by comparing the rate of change in BRET ratio caused by an unknown sample with the rate of change of BRET ratio caused by a purified preparation of the same esterase, phosphatase, lipase or other hydrolase enzyme, whose specific activity had been determined by another means, under the same or similar conditions. Many such purified preparations of enzymes are commercially available from a variety of suppliers such as Merck. Alternatively, the conversion rate of the substrate can be estimated in a parallel assay by omitting coelenterazine from the latter reaction and instead measuring the rate of increase in concentration of the unblocked fluorophore, using absorbance spectrometry. In the case of a blocked fluorescein group, such as fluorescein acetate, fluorescein phosphate or fluorescein laurate, one would use the published molar absorptivity of fluorescein at a given wavelength and the assay pH (e.g. as disclosed in Sjoback et al., 1995) subtracting any background absorption, to calibrate the rate of change in BRET ratio under the same assay conditions, as the number of moles of the sensor converted per minute. A calibration, once performed, could be applied to measurements taken at different times under similar or identical conditions.

If it was desired to express the enzyme activity in terms of specific activity (i.e. micromoles of substrate converted per minute per mg of protein) it would be necessary also to estimate the concentration of protein in the sample using any generally acceptable method, such as absorption at 280 nm, the Bradford protein assay, the Lowry protein assay, the bicinchoninic acid protein assay, or any of the published and or commercially available alternatives to or variations of these methods that are known to persons skilled in the art.

If it was desired to express the amount of an enzyme present in a sample, not in terms of activity but rather as a mass, it would be possible to calculate this using a typical or measured specific activity for a pure preparation of the enzyme of interest. For example, if, using the approach above, a particular esterase-containing sample supported a rate of change of BRET ratio in the assay that had been determined to be equivalent to 0.005 micromoles of substrate converted per minute and the specific activity of the pure esterase was known to be 100 micromoles of substrate converted per minute per milligram of protein then the amount of esterase present would be calculated as 0.005/100=0.00005 mg or 50 ng in whatever volume of sample had been used.

If it was desired to express this as a number of moles then one would apply the published molecular mass of the enzyme of interest. For example, in the case of pig liver carboxylesterase (M=163,000±15,000; Horgan et al., 1969) and using the arbitrary example values from above, the molar quantity of an esterase present would be estimated as approximately 50 ng/163,000 gM 0.3 femtomoles.

This application claims priority from Australian application no. 2017903420 filed 24 Aug. 2017, the entire contents of which are incorporated by reference herein.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Babaei et al. (2007) Vet. Res. Commun. 31: 419-425.
Banks and Paquette (1995) Bioconjugate Chem. 6:447-458.
Basu et al. (2011) J. Lipid Res. 52:826-832.
Bogin and Ziv (1973) Cornell Veterinarian 63: 666-676.
Chandran et al. (2005) J. Am. Chem. Soc. 127:1652-1653.
Day et al. (2004) Luminescence 19:8-20.
de Wet et al. (1987) Mol. Cell. Biol. 2987:725-737.
Egholm et al. (2005) Nature 365:566-568.
Förster (1948) Ann. Physik. 2:55-75.
Förster (1960) Rad. Res. Suppl. 2:326-339.
Greer and Szalay (2002) Luminescence 17:43-74.
Hall et al. (2012) ACS Chem. Biol. 7:1848-57.
Hastings (1996) Gene 173:5-11.
Herve et al. (2008) AAPS J. 10:455-472.
Horgan et al. (1969) Biochemistry 8: 2006-2013.
Hoy and Neave FK (1937) The Lancet 230:595-598.
Iglesias et al. (2016) J. Lipid Res. 57:131-141.
Kay (1935) The Lancet 225:1516-1518.
Lavis et al. (2006a) Chembiochem. 7:1151-1154.
Lavis et al. (2006b) ACS Chemical Biology 1:252-260.
Levine and Raines (2012) Chem. Sci. 3:2412-2420.
Loening et al. (2006) Protein Eng. Des. Sel. 19:391-400.
Loening et al. (2007) Nature Methods 4:641-643.
Lorenz et al. (1991) Proc. Natl. Acad. Sci. USA 88:4438-4442.
Martins (2015) Braz. J Microbiol. 46:207-217.
Oteng-Pabi et al. (2014) Chem. Commun. (Camb). 50:6604-6606.
Quiocho et al. (1997) Structure 5:997-1015.
Rankin et al. (2010) J. Dairy Sci. 93: 5538-5551.
Schechter and Berger (1967) Biochem. Biophys. Res. Commun. 27:157-162.
Schechter and Berger (1968) Biochem. Biophys. Res. Commun. 32:898-902
Singh et al. (1998) Chem. Commun. 455-456.
Sjoback et al. (1995) Spectrochim. Acta. A, Mol. Biomol. Spectrosc. 51: L7-L21.
Stoytcheva et al. (2012) Curr. Anal. Chem. 8:400-407.
Theile et al. (2013) Nat. Protoc. 8:1800-1807.
Verhaegen et al. (2002) Anal. Chem. 74:4378-4385
Viviani (2002) Cell. Mol. Life Sci. 59:1833-1850.
Xu et al. (1999) Proc. Natl. Acad. Sci. USA. 96:151-156.
Ż [symbol] dło-Dobrowolska et al., (2016) Org. Biomol. Chem. 14:9146-9150.
Zheng et al. (2004) Nucl. Acids Res. 32:e115.

1. A sensor molecule for detecting a hydrolase, the sensor molecule having a general formula selected from:
R¹-L-R²—B (I), or
B—R²-L-R¹ (II)
wherein
R¹ is a bioluminescent protein;
L is a linking element;
R² is a non-protein acceptor domain; and
B is a blocking group,
wherein R² bound to B comprises a hydrolysable bond and hydrolysis of the hydrolysable bond by the hydrolase produces a change in bioluminescence resonance energy transfer (BRET).

2. The sensor molecule of claim 1, wherein the blocking group stabilises the acceptor domain in a low-fluorescent or non-fluorescent state.

3. The sensor molecule of claim 1 or claim 2, wherein the blocking group comprises a phosphate containing moiety, sugar containing moiety, amino acid containing moiety, nucleotide, nucleoside, ester or ether.

4. The sensor molecule of any one of claims 1 to 3, wherein the linking element comprises an alkyl chain, glycol, ether, polyether, polyamide, polyester, peptide, polypeptide, amino acid or polynucleotide.

5. The sensor molecule of claim 4, wherein the linking element comprises a polypeptide.

6. The sensor molecule of claim 5, wherein R¹-L or L-R¹ are a single polypeptide.

7. The sensor molecule of claim 5 or claim 6, wherein the linking element comprises a cysteine residue and/or a lysine residue.

8. The sensor molecule of claim 7, wherein R² is attached to the linking element via the cysteine residue.

9. The sensor molecule of any one of claims 1 to 8, wherein R² is selected from an Alexa Fluor dye, Bodipy dye, Cy dye, fluorescein, dansyl, umbelliferone, fluorescent microsphere, luminescent microsphere, fluorescent nanocrystal, Marina Blue, Cascade Blue, Cascade Yellow, Pacific Blue, Oregon Green, Tetramethylrhodamine, Rhodamine, coumarin, BODIPY, resorufin, Texas Red, rare earth element chelates, or any combination or derivative thereof.

10. The sensor molecule of any one of claims 1 to 9, wherein R¹ is selected from a luciferase, a β-galactosidase, a lactamase, a horseradish peroxidase, an alkaline phosphatase, a β-glucuronidase or a β-glucosidase.

11. The sensor molecule of claim 10, wherein the luciferase is a Renilla luciferase, a Firefly luciferase, a Coelenterate luciferase, a North American glow worm luciferase, a click beetle luciferase, a railroad worm luciferase, a bacterial luciferase, a Gaussia luciferase, Aequorin, an Arachnocampa luciferase, or a biologically active variant or fragment of any one, or chimera of two or more, thereof.

12. The sensor molecule of any one of claims 1 to 11, wherein the hydrolase is an esterase, lipase, protease, phosphatase, nuclease, glycosidase, DNA glycosylases or an acid anhydride hydrolase.

13. The sensor molecule of any one of claims 1 to 12, wherein the separation and relative orientation of R¹ and R², in the presence and/or the absence of hydrolase, is within ±50% of the Förster distance.

14. The sensor molecule of claim 13, wherein the Förster distance of R¹ and R² is at least 4.0 nm.

15. The sensor molecule of claim 14, wherein the Förster distance of R¹ and R² is between about 4.0 nm and about 10 nm.

16. A method of detecting a hydrolase in a sample, the method comprising
i) contacting a sample with the sensor molecule of any one of claims 1 to 15 and claim 31; and
ii) detecting a change in BRET ratio, wherein the change in the BRET ratio corresponds to the presence of a hydrolase in the sample.

17. A method of detecting a hydrolase in a sample, the method comprising:
i) contacting a sample with a blocked non-protein acceptor domain having the structure B—R² to form a treated sample;
ii) contacting the treated sample with a compound of formula R¹-L or L-R¹ under conditions to cause attaching of R² to L; and
iii) detecting a change in BRET ratio, wherein the change in the BRET ratio corresponds to the presence of a hydrolase in the sample and the formation of a compound of formula R¹-L-R² or R²-L-R¹,
and wherein
R¹ is a bioluminescent protein;
L is a linking element;
R² is a non-protein acceptor domain; and
B is a blocking group and R² bound to B comprises a hydrolysable bond.

18. The method of claim 17, wherein R² comprises a cysteine specific electrophile or an amine specific electrophile.

19. The method of claim 17 or claim 18, wherein L comprises a cysteine and/or a lysine residue.

20. The method of any one of claims 18 to 21, further comprising determining the concentration of the hydrolase in the sample and/or activity of the hydrolase in the sample.

21. The method of any one of claims 18 to 22 which is performed on a microfluidic device.

22. The method of any one of claims 16 to 21, wherein the sample is any one of air, liquid, biological material or soil.

23. The method of claim 22, wherein the sample comprises a biological material selected from the group consisting of milk, blood, serum, sputum, mucus, pus and peritoneal fluid.

24. A variant bioluminescent protein comprising at least one less cysteine residue when compared to the corresponding naturally occurring protein.

25. The variant bioluminescent protein of claim 24 which lacks a cysteine residue at a position corresponding position 24 or position 73 of RLuc8 (SEQ ID NO: 50).

26. The variant bioluminescent protein of claim 24 which lacks a cysteine residue at a position corresponding to amino acid position 24 and position 73 of RLuc8 (SEQ ID NO: 50).

27. A polynucleotide encoding the variant bioluminescent protein of any one of claims 24 to 26.

28. A vector comprising the polynucleotide of claim 27.

29. A host cell comprising the polynucleotide of claim 27 and/or the vector of claim 28.

30. A process for producing a variant bioluminescent protein, the process comprising cultivating a host cell of claim 29 or a vector of claim 28 under conditions which allow expression of the polynucleotide encoding the protein, and recovering the expressed protein.

31. The sensor molecule of any one of claims 1 to 15, wherein the R¹ is the variant bioluminescent protein of any one of claims 24 to 26.


SEQUENCE LISTING (51 sequences)

SEQ ID NO: 1 - protein, 349 aa, Artificial Sequence
  wt-RLuc8
MRGSHHHHHHGMASMTGGQQMGRDLYDDDDKDRWGSEFMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 2 - protein, 349 aa, Artificial Sequence
  RLuc8Cys1
MRGSHHHHHHGMASMTGGQQMGRDLYDDDDKDRWGSECMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 3 - protein, 349 aa, Artificial Sequence
  RLuc8Cys2
MRGSHHHHHHGMASMTGGQQMGRDLYDCDDKDRWGSEFMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 4 - protein, 349 aa, Artificial Sequence
  RLuc8Cys3
MRGSHHHHHHGMASMTGCQQMGRDLYDDDDKDRWGSEFMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 5 - protein, 11 aa, Artificial Sequence
  linking element
CDDKDRWGSEF

SEQ ID NO: 6 - protein, 21 aa, Artificial Sequence
  linking element
CQQMGRDLYDDDDKDRWGSEF

SEQ ID NO: 7 - protein, 38 aa, Artificial Sequence
  linking element
MRGSHHHHHHGMASMTGGQQMGRDLYDDDDKDRWGSEF

SEQ ID NO: 8 - protein, 311 aa, Artificial Sequence
  wt-RLuc8(C24X)
  X (24)..(24): X is any amino acid other than a cysteine.
MASKVYDPEQRKRMITGPQWWARXKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 9 - protein, 311 aa, Artificial Sequence
  wt-RLuc8(C73Z)
  Z (73)..(73): Z is any amino acid other than a cysteine.
MASKVYDPEQRKRMITGPQWWARCKQMNVLDSFINYYDSEKHAENAVIFLHGNATSSYLWRHVVPHIEPVARZIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 10 - protein, 311 aa, Artificial Sequence
  wt-RLuc8(C24X.C73Z)
  X (24)..(24): X is any amino acid other than a cysteine.
  Z (73)..(73): Z is any amino acid other than a cysteine.
MASKVYDPEQRKRMITGPQWWARXKQMNVLDSFINYYDSEKHAENAVIFLHGNATSSYLWRHVVPHIEPVARZIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 11 - DNA, 1050 bp, Artificial Sequence
  nucleotide sequence encoding wt-RLuc8
atgcggggttctcatcatcatcatcatcatggtatggctagcatgactggtggacagcaaatgggtcgggatctgtacgacgatgacgataaggatcgatggggatccgaattcatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaa

SEQ ID NO: 12 - DNA, 1050 bp, Artificial Sequence
  nucleotide sequence encoding RLuc8Cys1
atgcggggttctcatcatcatcatcatcatggtatggctagcatgactggtggacagcaaatgggtcgggatctgtacgacgatgacgataaggatcgatggggatccgaatgcatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaa

SEQ ID NO: 13 - DNA, 1050 bp, Artificial Sequence
  nucleotide sequence encoding RLuc8Cys2
atgcggggttctcatcatcatcatcatcatggtatggctagcatgactggtggacagcaaatgggtcgggatctgtacgactgcgacgataaggatcgatggggatccgaattcatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaa

SEQ ID NO: 14 - DNA, 1050 bp, Artificial Sequence
  nucleotide sequence encoding RLuc8Cys3
atgcggggttctcatcatcatcatcatcatggtatggctagcatgactggttgccagcaaatgggtcgggatctgtacgacgatgacgataaggatcgatggggatccgaattcatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaa

SEQ ID NO: 15 - DNA, 29 bp, Artificial Sequence
  RLuc8Cys1-F
atggggatccgaatgcatggcttccaagg

SEQ ID NO: 16 - DNA, 29 bp, Artificial Sequence
  RLuc8Cys1-R
ccttggaagccatgcattcggatccccat

SEQ ID NO: 17 - DNA, 30 bp, Artificial Sequence
  RLuc8Cys2-F
ggatctgtacgactgcgacgataaggatcg

SEQ ID NO: 18 - DNA, 30 bp, Artificial Sequence
  RLuc8Cys2-R
cgatccttatcgtcgcagtcgtacagatcc

SEQ ID NO: 19 - DNA, 30 bp, Artificial Sequence
  RLuc8Cys3-F
ctagcatgactggttgccagcaaatgggtc

SEQ ID NO: 20 - DNA, 30 bp, Artificial Sequence
  RLuc8Cys3-R
gacccatttgctggcaaccagtcatgctag

SEQ ID NO: 21 - protein, 7 aa, Artificial Sequence
  High affinity substrate for mTG
WALQRPH

SEQ ID NO: 22 - protein, 7 aa, Artificial Sequence
  High affinity substrate for mTG
WELQRPY

SEQ ID NO: 23 - protein, 4 aa, Artificial Sequence
  Sortase recognition sequence
  X (3)..(3): X is any amino acid
LPXT

SEQ ID NO: 24 - protein, 6 aa, Artificial Sequence
  spacer sequence
GSSGGS

SEQ ID NO: 25 - protein, 6 aa, Artificial Sequence
  Spacer sequence
GGSGGS

SEQ ID NO: 26 - protein, 6 aa, Artificial Sequence
  Spacer sequence
GGTGGG

SEQ ID NO: 27 - protein, 6 aa, Artificial Sequence
  Spacer sequence
GGGGGT

SEQ ID NO: 28 - protein, 8 aa, Artificial Sequence
  Spacer sequence
LQGGTGGG

SEQ ID NO: 29 - protein, 8 aa, Artificial Sequence
  Specaer sequence
FEGGTGGG

SEQ ID NO: 30 - protein, 7 aa, Artificial Sequence
  Spacer sequence
GGSGGSL

SEQ ID NO: 31 - protein, 5 aa, Artificial Sequence
  High affinity substrate for mTG
  X (3)..(3): X is any amino acid
  Z (5)..(5): Z is Gly or Ala
LPXTZ

SEQ ID NO: 32 - protein, 349 aa, Artificial Sequence
  RLuc8Cys4
MRGSHHHHHHCMASMTGGQQMGRDLYDDDDKDRWGSEFMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 33 - protein, 349 aa, Artificial Sequence
  RLuc8Cys5
MCGSHHHHHHGMASMTGGQQMGRDLYDDDDKDRWGSEFMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 34 - protein, 724 aa, Artificial Sequence
  MBP(K239C)RLuc8
MRGSHHHHHHGMASMTGGQQMGRDLYDDDDKDRWGSEFMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSCVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGTGGFEMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 35 - DNA, 1050 bp, Artificial Sequence
  nucleotide sequence encoding RLuc8Cys4
atgcggggttctcatcatcatcatcatcattgcatggctagcatgactggtggacagcaaatgggtcgggatctgtacgacgatgacgataaggatcgatggggatccgaattcatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaa

SEQ ID NO: 36 - DNA, 1050 bp, Artificial Sequence
  nucleotide sequence encoding RLuc8Cys5
atgtgcggttctcatcatcatcatcatcatggtatggctagcatgactggtggacagcaaatgggtcgggatctgtacgacgatgacgataaggatcgatggggatccgaattcatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaa

SEQ ID NO: 37 - DNA, 2175 bp, Artificial Sequence
  nucleotide sequence encoding MBP(K239C)RLuc8
atgcggggttctcatcatcatcatcatcatggtatggctagcatgactggtggacagcaaatgggtcgggatctgtacgacgatgacgataaggatcgatggggatccgaattcatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagctgcgtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactggcggcggtaccggtggattcgaaatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaa

SEQ ID NO: 38 - DNA, 33 bp, Artificial Sequence
  RLuc8Cys4-F
tcatcatcatcatcattgcatggctagcatgac

SEQ ID NO: 39 - DNA, 33 bp, Artificial Sequence
  RLuc8Cys4-R
gtcatgctagccatgcaatgatgatgatgatga

SEQ ID NO: 40 - DNA, 35 bp, Artificial Sequence
  RLuc8Cys5-F
aaggagatatacatatgtgcggttctcatcatcat

SEQ ID NO: 41 - DNA, 35 bp, Artificial Sequence
  RLuc8Cys5-R
atgatgatgagaaccgcacatatgtatatctcctt

SEQ ID NO: 42 - DNA, 35 bp, Artificial Sequence
  MBP(K239C)-F
tggtccaacatcgactgcagcaaagtgaattatgg

SEQ ID NO: 43 - DNA, 35 bp, Artificial Sequence
  MBP(K239C)-R
aacatcgacaccagctgcgtgaattatggtgtaac

SEQ ID NO: 44 - protein, 38 aa, Artificial Sequence
  linking element containing a cysteine residue
MRGSHHHHHHGMASMTGGQQMGRDLYDDDDKDRWGSEC

SEQ ID NO: 45 - protein, 38 aa, Artificial Sequence
  Linking element containing a cysteine residue
MRGSHHHHHHGMASMTGGQQMGRDLYDCDDKDRWGSEF

SEQ ID NO: 46 - protein, 38 aa, Artificial Sequence
  Linking element containing a cysteine residue
MRGSHHHHHHGMASMTGCQQMGRDLYDDDDKDRWGSEF

SEQ ID NO: 47 - protein, 38 aa, Artificial Sequence
  Linking element containing a cysteine residue
MRGSHHHHHHCMASMTGGQQMGRDLYDDDDKDRWGSEF

SEQ ID NO: 48 - protein, 38 aa, Artificial Sequence
  Linking element containing a cysteine residue
MCGSHHHHHHGMASMTGGQQMGRDLYDDDDKDRWGSEF

SEQ ID NO: 49 - protein, 311 aa, Artificial Sequence
  RLuc
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 50 - protein, 311 aa, Artificial Sequence
  RLuc8
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 51 - protein, 311 aa, Artificial Sequence
  RLuc2
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETVLPSKIMRKLEPEEFAAYLEPFKEKGEVRRPTLSWPREIPLVKGGKPDVVAIVRNYNAYLRASDDLPKMFIESDPGFFSNAIVEGAKKFPNTEFVKVKGLHFSQEDAPDEMGKYIKSFVERVLKNEQ